(19) United States
(12) Patent Application Publication
(10) Pub. No.: US 2026/0083752 A1
(43) Pub. Date: Mar. 26, 2026

(54) PHARMACEUTICAL METHODS, COMPOSITIONS AND COMBINATIONS

(71) Applicant: Regents of the University of Minnesota, Minneapolis, MN (US)

(72) Inventors: Darko Bosnakovski, Minneapolis, MN (US); Erdong Wei, Minneapolis, MN (US)

(73) Assignee: Regents of the University of Minnesota, Minneapolis, MN (US)

(21) Appl. No.: 19/334,371

(22) Filed: Sep. 19, 2025

Related U.S. Application Data
(60) Provisional application No. 63/697,288, filed on Sep. 20, 2024.

(51) Int. Cl. A61K 31/553; A61K 31/352; A61K 31/404; A61K 31/4184; A61K 31/423; A61K 31/4412; A61K 31/4439; A61K 31/444; A61K 31/454; A61K 31/47; A61K 31/4745; A61K 31/496; A61K 31/501; A61K 31/5025; A61K 31/506; A61K 31/519; A61K 31/5377; A61K 31/635; A61K 47/54; A61K 47/55; A61P 35/00

(52) U.S. Cl. CPC A61K 31/553; A61K 31/352; A61K 31/404; A61K 31/4184; A61K 31/423; A61K 31/4412; A61K 31/4439; A61K 31/444; A61K 31/454; A61K 31/47; A61K 31/4745; A61K 31/496; A61K 31/501; A61K 31/5025; A61K 31/506; A61K 31/519; A61K 31/5377; A61K 31/635; A61K 47/545; A61K 47/55; A61P 35/00

(57) ABSTRACT

Disclosed herein are methods for treating cancer in an animal in need thereof, comprising administering to the animal a p300/CBP (p300 and/or CBP) modulator or a pharmaceutically acceptable salt thereof and a senotherapeutic (e.g., a senolytic or senomorphic) or a pharmaceutically acceptable salt thereof. Also disclosed are pharmaceutical compositions and pharmaceutical combinations, comprising a p300/CBP (p300 and/or CBP) modulator or a pharmaceutically acceptable salt thereof and a senotherapeutic (e.g., a senolytic or senomorphic) or a pharmaceutically acceptable salt thereof.

### CROSS-REFERENCE TO RELATED APPLICATION

This application claims priority to U.S. Provisional Application No. 63/697,288 that was filed on Sep. 20, 2024. The entire content of the applications referenced above is hereby incorporated by reference herein.

### GOVERNMENT FUNDING

This invention was made with government support under HT9425-23-1-0456 awarded by the Defense Health Agency, Medical Research and Development Branch. The government has certain rights in the invention.

### BACKGROUND OF THE INVENTION

Ewing Sarcoma (ES), the second most common form of pediatric bone cancer, primarily affects individuals at a median age of 15 years and occurs with an incidence of approximately three cases per million annually [1, 2]. Over the last fifty years, improvements in diagnosis, surgery, chemotherapy, and radiation have increased survival rates to nearly 70% for localized ES, yet the prognosis for patients with metastatic or recurrent forms remains challenging, with a five-year survival rate still below 25% [3, 4]. This disparity underscores an urgent need for new therapeutic targets and strategies.

Accordingly, there is a need for methods, compositions and combinations for treating cancer, including methods compositions and combinations that include multiple therapeutic agents.

### SUMMARY OF THE INVENTION

One embodiment provides a method for treating cancer in an animal (e.g., a mammal such as a human) in need thereof, comprising administering (e.g., co-administering) to the animal a p300/CBP (p300 and/or CBP) modulator or a pharmaceutically acceptable salt thereof and a senotherapeutic (e.g., a senolytic or senomorphic) or a pharmaceutically acceptable salt thereof.

One embodiment provides a method for treating cancer in an animal (e.g., a mammal such as a human) in need thereof, comprising administering (e.g., co-administering) to the animal a p300/CBP (p300 and/or CBP) modulator or a pharmaceutically acceptable salt thereof and a senolytic or a pharmaceutically acceptable salt thereof.

One embodiment provides a method pharmaceutical composition or pharmaceutical combination, comprising a p300/CBP (p300 and/or CBP) modulator or a pharmaceutically acceptable salt thereof and a senotherapeutic (e.g., a senolytic or senomorphic) or a pharmaceutically acceptable salt thereof.

In one embodiment the p300/CBP (p300 and/or CBP) modulator is a p300/CBP inhibitor

In one embodiment the p300/CBP (p300 and/or CBP) modulator is a p300/CBP degrader.

In one embodiment the p300/CBP (p300 and/or CBP) modulator is a p300/CBP (p300 and/or CBP) histone acetyltransferase (HAT) inhibitor or p300/CBP (p300 and/or CBP) bromodomain inhibitor.

In one embodiment the p300/CBP (p300 and/or CBP) modulator or a pharmaceutically acceptable salt thereof is B029-2, XP-524, A-485, CCS1477, NE02734, B026, FT-7051, or a compound (e.g., a p300 and/or CBP inhibitor) or a pharmaceutically acceptable salt thereof as described in WO 2016/044770.

In one embodiment the p300/CBP (p300 and/or CBP) modulator (e.g., inhibitor) is a compound of formula (I) (iP300w):

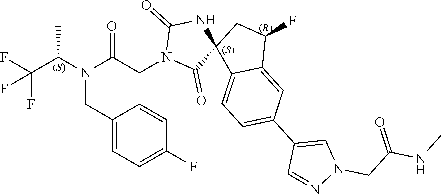

or a pharmaceutically acceptable salt thereof to the animal.

In one embodiment the p300/CBP (p300 and/or CBP) modulator or a pharmaceutically acceptable salt thereof is a PROTAC.

In one embodiment the PROTAC is dCBP-1 or JQAD1.

In one embodiment the PROTAC is BT-02C, dCBP-1, or JQAD1.

In one embodiment the senotherapeutic (e.g., a senolytic or senomorphic) or a pharmaceutically acceptable salt thereof is Dasatinib, Fisetin, Navitoclax, or Quercetin.

In one embodiment the senotherapeutic (e.g., a senolytic or senomorphic) is a tyrosine kinase inhibitor (TKI) or a pharmaceutically acceptable salt thereof, selected from the group consisting of Dasatinib, Imatinib, Nilotinib, Bosutinib, Ponatinib, or Asciminib. Sunitinib, Sorafenib, Axitinib, Lenvatinib, Gefitinib, Erlotinib, Vandetanib, Cabozantinib, Pazopanib, Crizotinib, Ceritinib, Brigatinib, Larotrectinib, Entrectinib, Regorafenib, Osimertinib, Alectinib, Trametinib, Neratinib, and Tucatinib.

In one embodiment the senotherapeutic (e.g., a senolytic or senomorphic) is a PI3K (phosphoinositide 3-kinase) inhibitor or a pharmaceutically acceptable salt thereof, selected from the group consisting of Idelalisib, Alpelisib, Copanlisib, Duvelisib, Buparlisib, Pictilisib, Apitolisib, Dactolisib, Taselisib, Omipalisib, and Voxtalisib.

In one embodiment the cancer is a cancer driven by an oncogene that is dependent on p300/CBP activity (e.g., the method to reduce the proliferation of oncogene-driven cancer cells).

In one embodiment the oncogene is EWSR1::FLI1, EWSR1::ERG, AML::ETO, ATXN1::DUX4, CIC::DUX4, CIC::FOXO4, CIC::NUTM1, CIC::LEUTX, MYB::NFIB, RUNX1::ETO, PAX3::FOXO1, PAX3::FOXO4, PAX3::INO80D, PAX3::AFX, PAX3::NCOA1, PAX3::NCOA2, BCOR::CCNB3, BCOR::MAML3, BCOR::ITD, PAX7::FOXO1, ASPSCR1::TFE3, EWSR1::CREB1, EWSR1::ATF1, PAX3::MAML3, MECT1::MAML2, BRD4::NUTM1, BRD3::NUTM1, NSD3::NUTM1, YWHAE::NUTM2, EWSR1::WT1, EP300::BCOR, FUS::ERG, FUS::CREB3L1, FUS::CREB3L2a, EWSR1::CREB3L1, EWSR1::CREB3L2, FUS::CREB3L2, FUS::DDIT3a, NAB2::STAT6, VGLL2::CITED2, SS18::SSX1, SS18::SSX2, SS18::SSX4, SS18L1::SSX1, BCOR::MAML3, NUTM2A::CIC, YWHAE::NUTM2B, EWSR1::SP3, PAX8::PPARG, RUNX1::ET, TMPRSS2::ERG, TMPRSS2::ETV1, or TMPRSS2::ETV4.

In one embodiment the cancer is a pediatric sarcoma, CIC-rearranged sarcoma or Ewing sarcoma.

In one embodiment the cancer is a pediatric sarcoma, CIC-rearranged sarcoma, Ewing sarcoma, or CIC-DUX4 sarcoma.

One embodiment provides a pharmaceutical composition or pharmaceutical combination comprising a p300/CBP (p300 and/or CBP) modulator or a pharmaceutically acceptable salt thereof and a senotherapeutic (e.g., a senolytic or senomorphic) or a pharmaceutically acceptable salt thereof for medical treatment.

One embodiment provides the use of a pharmaceutical composition or pharmaceutical combination comprising a p300/CBP (p300 and/or CBP) modulator or a pharmaceutically acceptable salt thereof and a senotherapeutic (e.g., a senolytic or senomorphic) or a pharmaceutically acceptable salt thereof for the treatment of cancer.

One embodiment provides a pharmaceutical composition or pharmaceutical combination comprising a p300/CBP (p300 and/or CBP) modulator or a pharmaceutically acceptable salt thereof and a senotherapeutic (e.g., a senolytic or senomorphic) or a pharmaceutically acceptable salt thereof for use in the manufacture of a medicament for the treatment of cancer.

### BRIEF DESCRIPTION OF DRAWINGS

The patent or application file contains at least one drawing executed in color. Copies of this patent or patent application publication with color drawings will be provided by the Office upon request and payment of the necessary fee.

FIGS. 1A-1C show that siRNA-mediated knockdown of P300/CBP unveils a pivotal role in ES. FIG. 1A bar graphs show the significant reduction in cell viability across SKES1, A4573, and TC71 ES cell lines after siRNA-mediated knockdown of P300/CBP at 72 hours. The data represent mean±SEM; **p<0.001, ****p<0.0001 by two-way ANOVA, n=4. Results are presented as percentage viability, normalized to the control sample. FIG. 1B show that western blot analysis confirms the knockdown of P300/CBP at the protein level and reveals decreased levels of acetylated histone markers (acH3K18 and acH3K27) after 48 hours of siRNA treatment. “Cont” refers to untreated cells, “siCont” indicates cells treated with scramble siRNA, and “siCBP/P300” refers to cells treated with siRNA targeting both CBP and P300. FIG. 1C shows RT-qPCR analysis at 48 hours following P300/CBP knockdown shows decreased expression of EWS::FLI1 regulated genes (NR0B1, MEIS1, c-MYC, ID2) in SKES1 and A4573 cell lines. Silencing P300 or CBP individually led to a reduction in the target genes, with a more pronounced effect observed upon concurrent silencing of both. Data are presented as mean SEM; *p<0.05, by two-way ANOVA. Results are presented as relative expression to B2M (n=6).

FIGS. 2A-2I show successful pharmacological targeting of ES using iP300w. FIG. 2A bar graphs show the dose-dependent inhibition of viability in ES cell lines (SKES1, A673, A4573, and TC71) compared to normal human myoblast cells (LHCN) following iP300w treatment for 48 hours. The ATP assay data are represented as mean±SEM; p<0.05, by two-way ANOVA. Results are presented as fold difference compared to control (untreated group at 48 hours) (n=3). FIG. 2B shows SKES1 cell morphology after 48 hours of treatment with 1 μM iP300w (scale bar 100 μm). FIG. 2C shows that immunofluorescence for Ki67 (red) expression in SKES1 cells indicates cell-cycle arrest after 48 hours of iP300w treatment (1 μM). DAPI (blue) was used to stain the nuclei. FIG. 2D shows western blot analysis for EWS::FLI1, P300, CBP, acH3K9, acH3K18 and acH3K27, in SKES1 and TC71 cell lines treated with 1 μM iP300w for 4 and 12 hours. FIG. 2E bar graphs show RT-qPCR results illustrating unchanged EWS::FLI1 expression following P300/CBP inhibition, with a significant reduction in EWS::FLI1 target gene expression (NKX2.2 and CMYC) at 24 hours post-treatment. Data were normalized to B2M. The data represent the mean±SEM, p<0.05, by t-test. Results are presented as fold differences compared to the control (untreated group at 24 hours) (n=6). FIG. 2F shows gross morphology of tumors from treated (iP300w, 5.6 mg/kg daily) and control mice at the terminal point of the experiment (day 14). FIG. 2G shows graphs that display tumor volume and weight measurements, demonstrating significant reductions in tumor size and mass following treatment. Data are presented as mean±SEM; ***p<0.001, ****p<0.0001, by t-test (control, n=6; treatment, n=7). FIG. 2H shows western blot for acetylated H3K18 and H3K27 in tumor samples presented in FIG. 2F. FIG. 2I shows RT-qPCR analysis of EWS::FLI1, its target genes, senescence-related genes, and cell cycle genes in the xenografts from experiment (FIG. 2F). Data were normalized to B2M and compared to the control group using a t-test. The data represent mean±SEM; *p<0.05, **p<0.01, ***p<0.001, ****p<0.0001, (control, n=6; treatment, n=7).

FIGS. 3I-3J show P300/CBP inhibition and EWS::FLI1 knockdown yield comparable transcriptional changes in ES. FIG. 3A shows a heatmap that illustrates the gene expression profiles of ES cells treated with iP300w (1 μM) for 4 hours, with hierarchical clustering indicating significant concordance among different ES samples. FIG. 3B shows a Venn diagram that illustrates the overlap of DEGs in ES cells post P300/CBP inhibition, with a predominant downregulation of gene expression. FIG. 3C shows a Venn diagram of DEGs in three ES cell lines following EWS::FLI1 knockdown that reveals both common and unique DEGs across the cell lines [23]. FIG. 3D shows a comparative Venn diagram that illustrates the overlap between DEGs in iP300w-treated cells and EWS::FLI1 knockdown cells. FIG. 3E shows the UpsetR comparative analysis that illustrates the overlap of DEGs between ES cell lines and non-ES cell lines treated with iP300w. FIG. 3F shows GSEA enrichment analyses that revealed significant enrichment (p=8.54e-52) of EWS-FLI1 related pathways (Reactome Riggi) in iP300w-treated ES cell lines compared to other cancer cell lines. The EWS-FLI1 related genes in the “Reactome Riggi” were identified through the overexpression of EWS-FLI1 in human mesenchymal stem cells. FIG. 3G shows a volcano plot illustrating the distribution of DEGs influenced by the treatment, with EWS::FLI1 binding sites within 50 KB of the promoter region, Canonical GGAA microsatellite EWS-ETS activation sites were identified through ChIP-seq and selected based on their presence in at least 15 out of 18 ES cell lines. [23]. FIG. 3H shows GSEA enrichment analyses that reveals significant enrichment (p=2.18e-4) of cell cycle checkpoints in iP300w-treated ES cell lines compared to other cancer cell lines (Other). FIG. 3I shows KEGG pathway enrichment analyses on DEG in iP300w treated ES cells (4 hours). FIG. 3J shows KEGG pathway analyses conducted on DEGs following EWS::FLI1 knockdown in ES cell lines. Note that iP300w and EWS::FLI1 knockdown both affect the cell cycle and induce cell senescence.

FIGS. 4A-4E show P300/CBP-related genes are linked to poor prognosis in patients with ES. FIG. 4A shows a NMF clustering consensus map categorizing 142 ES patients into two distinct groups based on the expression patterns of iP300w-induced DEGs. FIG. 4B shows a heatmap that illustrates the expression profiles of the top five genes from each group identified by NMF clustering. FIG. 4C shows Kaplan-Meier survival curves that reveal significant differences in survival outcomes between the two patient groups identified through NMF clustering. Additional analyses include survival stratification by median age, as well as metastatic and relapse states. FIG. 4D shows a Cox regression analysis presenting the top five and bottom five hazard ratio genes significantly associated with overall survival among the 74 identified genes. FIG. 4E shows the Lasso Cox regression model that correlates gene expression with overall survival, assigning risk scores based on gene expression levels. Higher risk scores are identified as significant risk factors.

FIGS. 5A-5I show P300/CBP inhibition triggers senescence in ES cancer cell lines. FIG. 5A shows β-Gal and H&E staining in SKES1 cells at 24 and 72 hours of treatments with iP300w. FIG. 5B shows gene expression analysis that illustrates changes in proliferation and senescence markers in SKES1 cells following 1, 3, and 7 days of treatment with 1 μM iP300w. Data were normalized to B2M and are presented as fold change compared to the control group. The data represent mean±SEM; p<0.05, by one-way ANOVA (n=6). FIG. 5C shows immunostaining that illustrate a complete absence of LMNB1 (red) expression in iP300w-treated cells after 72 hours. Phalloidin (green) highlights cell morphology, and DAPI (blue) stains the nuclei. Scale bar: 50 μm. FIG. 5D shows immunostaining for P15 in cells treated with iP300w for 72 hours. FIG. 5E shows RT-qPCR analysis of senolytics target gene expression following 24 and 72 hours of iP300w (1 μM) treatment. Data were normalized to B2M and are presented as fold change compared to the control group. The data represent mean SEM; *p<0.05, by one-way ANOVA (n=6). FIG. 5F shows ATP assays that illustrate cell viability of SKES1 with different senolytics (1 μM) used alone or combined with iP300w (1 μM) following 72 hours of treatment. The data represent mean±SEM; **p<0.01, ****p<0.0001 by one-way ANOVA. Results are presented as fold change compared to the control group (n=4). FIG. 5G shows synergy scores between iP300w and various senolytics (Dasatinib, Fisetin, Navitoclax, and Quercetin) calculated using the Highest Single Agent (HSA) model via SynergyFinder Plus. The 3D plots display synergy scores across different concentrations of senolytics (X-axis: 0 μM, 0.01 μM, 0.1 μM, 1 μM, 10 μM, 50 μM) combined with iP300w (Y-axis: 0 μM, 1 μM). The Z-axis represents the synergy score, with positive values indicating synergy and negative values indicating antagonism. The strongest synergistic effects were observed with Dasatinib, while other senolytics demonstrated varying levels of synergy or antagonism. FIG. 5H shows FACS analysis of Annexin V staining (histogram) in SKES1 cells following 72 hours of treatment with iP300w (1 μM), Dasatinib (1 μM), Dasatinib+iP300w, and Doxorubicin (1 μM). FIG. 5I shows quantification of Annexin V staining. Data are presented as log 10 fluorescence intensity. The data represent mean±SEM; *p<0.05, ****p<0.0001 by one-way ANOVA (n=3).

FIGS. 6A-6E show EWS::FLI1 regulates LMNB1 and P15 and cellular senescence in ES. FIG. 6A shows ChIP-Seq analysis that reveals that EWS::FLI1 binds to regulatory elements of LMNB1 and P15 in the SKNMC cell line [8]. Specifically, EWS::FLI1 targets the repetitive GGAA motif in the regulatory elements of LMNB1 and a single motif in P15. EWS::FLI1 binding sites in LMNB1 are associated with P300 and acetylation of H3K27. FIG. 6B shows transcriptional levels (RNA-Seq) of LMNB1 and P15 following EWS::FLI1 knockdown (96 hours) reveals reduction of LMNB1 and induction of P15. FIG. 6C shows RT-qPCR for LMNB1 and P15 expression 48 hours post P300/CBP knockdown. Data were normalized to B2M and are presented as fold change compared to the control group. The data represent mean±SEM; p<0.05 by t-test (n=4). FIG. 6D shows ChIP-Seq data that illustrates EWS::FLI1 binding in LMNB1 and P15 regulatory elements in MSC overexpressing EWS::FLI1 [16]. FIG. 6E shows LMNB1 and P15 expression (RNA-Seq) in MSC overexpressing EWS::FLI1 and GFP control cells. Data are presented as mean expression levels. The data represent mean±SEM; p<0.05 by t-test (n=4).

FIGS. 7A-7D show the restoration of LMNB1 and P15 expression in iP300w-treated cells delays senescence and induces apoptosis. FIG. 7A shows 3-Gal staining performed on SKES1 cells treated with iP300w (1 μM) for 48 hours, as well as on control untreated cells. LMNB1 refers to SKES1 cells that constitutively overexpress LMNB1 from a viral construct, while siP15 denotes SKES1 cells with P15 knocked down. LMNB1+siP15 refers to the condition where LMNB1 is expressed and P15 is knocked down for 48 hours. FIG. 7B shows quantification of 3-Gal positive cells presented in FIG. 7A. The data represent mean±SEM; *p<0.05, ***p<0.001 by one-way ANOVA (n=3). FIG. 7C shows Annexin V staining (histogram) of SKES1 cells in the condition described above. FIG. 7D shows quantification of intensity of Annexin V staining. The data represent mean±SEM; *p<0.05, ****p<0.0001 by one-way ANOVA (n=3).

FIG. 8 shows the proposed mechanism by which EWS::FLI1 regulates LMNB1 in ES cancer cell lines. In proliferative ES cells, the EWS::FLI1/P300 complex induces LMNB1 by binding to its enhancer motif containing 14 GGAA repeats. Inhibition of P300/CBP by iP300w treatment suppresses EWS::FLI1 transcriptional activity, leading to reduced LMNB1 transcription. Consequently, SA-β-Ga and other early senescence markers are induced. In a subset of ES cancer cell lines with non-functional P53, EWS::FLI1 binds to a single GGAA motif in the regulatory elements of P15, independent of P300, and represses its transcription. Following treatment with iP300w and subsequent LMNB1 suppression, P15 is rapidly induced, contributing to the induction of senescence in ES cells.

FIGS. 9A-9E show the effect of iP300w on ES cell lines in vitro and in vivo. FIG. 9A shows the comparison of cell viability reduction between ES cell lines (SKES1, CHLA9, CHLA25) and osteosarcoma cell lines (U2OS, 143B, SJSA-1, G292) after 48 hours of treatment with 0.1 and 1 μM iP300w. Data are presented as mean±SEM; p<0.05 by two-way ANOVA (n=3). FIG. 9B shows representative Ki-67 (red) staining in ES cell lines (TC71, A4573, A673) after 48 hours of 1 μM iP300w treatment. Nuclei are stained with blue (DAPI). FIG. 9C shows a RT-qPCR analysis that illustrates the changes in EWS::FLI1 target genes in ES cells following 24 hours of treatment with 1 μM of iP300w. The data represent mean±SEM; *p<0.05 by two-way ANOVA (n=6). FIG. 9D shows gene expression changes (RNA-Seq) in ES cells 4 hours post-iP300w treatment. Data represent count, *p<0.05 by t-test (n=2). FIG. 9E shows RT-qPCR analysis of EWS::FLI1 target genes in SKES1 xenografts following 14 days of iP300w treatment (5.6 mg/kg daily). Data are presented as mean±SEM and analyzed using a t-test; *p<0.01, **p<0.001, ***p<0.0001 (n=6).

FIGS. 10A-10D show dynamic tumor ES tumor development and toxicity assessment of iP300w. FIG. 10A shows tumor volume in mm³ measured at day 14, day 17, and day 20 in the iP300w treatment (4.2 mg/kg daily). Data are analyzed using two-way ANOVA and presented as mean±SEM; *p<0.05 (n=5). FIG. 10B shows line graph illustrating body weight measurements for control and iP300w-treated (4.2 mg/kg daily) groups over a 20-day period. Data were analyzed using two-way ANOVA and are presented as mean±SEM; no significant differences were observed between the groups. FIG. 10C shows bar graphs illustrating serum levels of BUN, creatinine, Na, K, osmolality, ALP, ALT, and glucose in treated mice compared to controls. Data are analyzed using t-test and presented as mean±SEM; **p<0.01 (n=6). FIG. 10D shows representative H&E images showing liver and kidney tissues from treated and control mice.

FIGS. 11A-11C show PCA clustering of iP300w treated ES cell lines. FIG. 11A shows PCA clustering of transcriptional profiles (RNA-seq) from ES cancer cell lines (A4573, A673, TC71 and SKES1) treated with iP300w for 4 hours. PCA clustering of ES and non ES cancer cell line treated with iP300w for 4 hours. FIG. 11B shows PCA clustering of ES and non ES cancer cell line (143B, A204, Fuji, G292, HOS, Kitra-SRS, MG-63, SJSA-1, T47, U2OS) treated with iP300w for 4 hours. FIG. 11C shows ATP assay comparing viability of SKES1 ES cell line to other cancer cell lines (FUJI, A204, T47D, HOS) after 24 hours of iP300w treatment. Data were analyzed using two-way ANOVA and are presented as mean SEM; p<0.05 (n=3).

FIG. 12 shows Lasso Cox regression analysis for identifying survival-associated genes. The upper panel shows the coefficient profiles of selected genes as a function of the regularization parameter (log 2(lambda)) [71]. As lambda increases, the coefficients of more genes are shrunk towards zero, with a subset of genes (TSKU, IKZF2, NTN4, IL20RB, SMAD9, NDST4, NKX2-2, ZBTB16) remaining significant at the optimal lambda value (λ=0.11). The lower panel presents the Partial Likelihood Deviance as a function of log 2(lambda), where the red dotted line indicates the optimal lambda value (λ=0.11) selected based on the minimum deviance. Error bars represent the standard errors of the deviance.

FIGS. 13A-13D show P300/CBP inhibition triggers senescence in ES cell lines with (A4573 and CHLA9) and without (TC71 and CHLA25) functional P53. FIG. 13A shows β-Gal and H&E staining of TC71 cells following iP300w (1 μM) treatment for 24 and 72 hours. FIG. 13B shows 3-Gal staining in CHLA25, A4573 and CHLA9 cells after 72 hours iP300w treatment. FIG. 13C shows gene expression analysis that reveals changes in proliferation and senescence markers in TC71, CHLA25, A4573, and CHLA9 cells following 1, 3, and 7 days of treatment with 1 μM iP300w. Data were normalized to B2M and are presented as log 2 fold change compared to the control group. Data were analyzed using two-way ANOVA and are presented as mean±SEM; p<0.05. Note that LMNB1 suppression occurs in all cell lines, while P15 induction is observed only in ES cell lines with reported non-functional P53 (TC71 and CHLA25). FIG. 13D shows immunostaining for LMNB1 and P15 in iP300w treated cells after 72 hours of incubation correlated with the gene expression analyses.

FIGS. 14A-14H show P300/CBP inhibition triggers senescence in ES cell lines harboring EWS::ERG translocation. FIG. 14A shows ATP assay on EWS::FLI1 cell line (SKES1) and EWS::ERG cell lines (CADO-ES1 and COGE352) after 48 hours of treatment with 1 μM iP300w. The data represent mean±SEM; *p<0.05, by two-way ANOVA (n=6). FIG. 14B shows immunostaining illustrating changes in Ki67 (red) expression after 72 hours iP300w treatment. FIG. 14C shows gene expression analysis illustrating changes in proliferation and senescence markers in CADO-ES1 cells following 1 day, 3 days, and 7 days of treatment with 1 μM iP300w. Data were normalized to B2M and are presented as log 2 fold change compared to the control group. Data are analyzed using two-way ANOVA and presented as mean±SEM; * p<0.05. FIG. 14D shows β-Gal staining in CADO-ES1 cells following 72 hours of P300/CBP inhibition. FIG. 14E shows immunostaining for LMNB1 and P15 in CADO-ES1 cells after 72 hours iP300w treatment. FIG. 14F shows gene expression analysis illustrating changes in proliferation and senescence markers in COGE352 cells following 1 day, 3 days, and 7 days of treatment with 1 μM iP300w. Data were normalized to B2M and are presented as log 2 fold change compared to the control group. Data are analyzed using two-way ANOVA and presented as mean±SEM; * p<0.05. FIG. 14G shows β-Gal staining in COGE352 cells at 72 hours of treatment. FIG. 14H shows immunostaining for LMNB1 and P15 in 72 hours iP300w treated COGE352 cells.

FIGS. 15A-15G show senolytic screening, cell viability, and apoptosis in ES cell lines treated with iP300w. FIG. 15A shows RNA-seq analysis of senolytic targets following 4 hours of treatment of SKES1 and TC71 with 1 μM iP300w. Data are analyzed using two-way ANOVA and presented as mean±SEM; * p<0.05, (n=2). FIG. 15B shows ATP assays that illustrate SKES1 cell viability at different concentrations (0.01 μM, 0.1 μM, 1 μM, 10 μM, 20 μM, and 50 μM) of senolytics, used alone or combined with 1 μM iP300w, with data collected after 72 hours of treatment. Data are analyzed using two-way ANOVA and presented as mean SEM; * p<0.05 (n=4). FIG. 15C shows ATP assays illustrating TC71 cell viability at different concentrations (0.01 μM, 0.1 μM, 1 μM, 10 μM, 20 μM, and 50 μM) of senolytics, used alone or combined with 1 μM iP300w, with data collected after 72 hours of treatment. Data are analyzed using two-way ANOVA and presented as mean±SEM; * p<0.05 (n=4). FIG. 15D shows ATP assays that illustrate TC71 cell viability with different senolytics (1 μM) used alone or combined with 1 μM iP300w for 72 hours. Data is presented as mean±SEM; two-way ANOVA, **p<0.001, ****p<0.0001 (n=4). FIG. 15E shows synergy scores between iP300w and various senolytics (Dasatinib, Fisetin, Navitoclax, and Quercetin) were calculated using the Highest Single Agent (HSA) model via SynergyFinder Plus. The 3D plots display synergy scores across different concentrations of senolytics (X-axis: 0 μM, 0.01 μM, 0.1 μM, 1 μM, 10 μM, 50 μM) combined with iP300w (Y-axis: 0 μM, 1 μM). The Z-axis represents the synergy score, with positive values indicating synergy and negative values indicating antagonism. FIG. 15F shows FACS analyses for Annexin V positive SKES1 cells following 72 hours of treatment with iP300w (1 μM), Dasatinib (1 μM), Dasatinib+iP300w, and Doxorubicin (1 μM). FIG. 15G shows quantification of Annexin V staining. Data are presented as the percentage of positive cells. The data represent mean±SEM; *p<0.05, ****p<0.0001 by one-way ANOVA (n=3).

FIGS. 16A-16E show that EWS::FLI1 and EWS::ERG bind to regulatory elements of senescence-related genes. FIG. 16A shows ChIP-Seq analysis that explores EWS::FLI1 binding sites, H3K27ac and P300 in the regulatory elements of LMNB1, P15, P16, TP53 and P21 in SKNMC before and after EWS::FLI1 knockdown. FIG. 16B shows ChIP-Seq data that illustrates EWS::FLI1 and acH3K27 binding sites in LMNB1, P15, P16, TP53 and P21 in MSC overexpressing EWS::FLI1. FIG. 16C shows a ChIP-Seq analysis that illustrates binding sites in the regulatory elements of LMNB1, P15, P16, TP53, and P21 for EWS::FLI1 in SKES1 and EWS::ERG in TC32. FIG. 16D shows transcriptional levels (RNA-Seq) of P16, TP53 and P21 following EWS::FLI1 knockdown (96 hours). FIG. 16E shows P16, TP53 and P21 expression (RNA-Seq) in MSC overexpressing EWS::FLI1 and GFP control cells. Data are presented as mean expression levels±SEM; p<0.05 by t-test (n=4).

FIGS. 17A-17C show LMNB1 overexpression and P15 knockdown in senescence-induced SKES1 cells. FIG. 17A shows RT-qPCR analysis that illustrates constitutive LMNB1 overexpression and P15 knockdown in SKES1 cells. LB1 refers to SKES1 cells that constitutively overexpress LMNB1 from a viral construct, while siP15 denotes SKES1 cells with P15 knocked down. LMNB1+siP15 refers to the condition where LMNB1 is expressed and P15 is knocked down for 48 hours. Cells were treated with iP300w (1 μM) for 48 hours. The data represent mean±SEM, analyzed using two-way ANOVA; p<0.05 (n=3). FIG. 17B shows FACS analyses of Annexin V staining for SKES1 cells in the condition described above. FIG. 17C shows quantification of percent of Annexin V positive cells. Data are analyzed using one-way ANOVA and presented as mean±SEM; ***p<0.001, ****p<0.0001.

FIG. 18 shows original images of Western blots.

FIG. 19 shows a listing of primers and probes.

FIGS. 20A-20C show P300/CBP inhibition reduces viability, suppresses proliferation, and induces senescence-associated features in CDS cells. FIG. 20A shows ATP assay after 72 h treatment with iP300w, BMS, CCS1477, NEO2734, or ISOXDUAL across 0.001-10 μM. Values are shown as mean±SEM, with statistical significance indicated by *p<0.05 (one-way ANOVA, compared to corresponding vehicle control). FIG. 20B shows Ki67 immunofluorescence after 72 h treatment. FIG. 20C shows senescence-associated 3-galactosidase staining after 72 h treatment. FIG. 20D shows LMNB1 immunofluorescence after 72 h treatment. Data represent n=3 independent experiments.

FIGS. 21A-21D show that the combination of iP300w and senolytics enhances loss of viability and prevents proliferative recovery in CDS cells. FIG. 21A shows ATP viability assays following 72 h iP300w induction at 0, 0.03, or 0.1 μM and a subsequent 72 h co-treatment phase with the same iP300w concentration plus graded Dasatinib or Navitoclax. Values are shown as mean±SEM, with statistical significance indicated by *p<0.05 (two-way ANOVA, internal comparisons to controls). FIG. 21B shows SynergyFinder Plus analysis of the combination matrices under the HSA model. FIG. 21C shows sequential assay incorporating 72 h iP300w induction, 72 h co-treatment with iP300w and senolytics agents, and 72 h drug-free recovery; EdU pulse at 24 h of recovery for 8 h; endpoint EdU and Ki67 readouts. FIG. 21D shows LMNB1 and DAPI staining under the same sequential conditions. Data represent n=4 independent experiments

### DETAILED DESCRIPTION

ES is defined by chromosomal translocations that form fusion genes encoding aberrant transcription factors crucial for pathogenesis. The predominant translocation, t(11;22)(q24;q12), present in about 85% of cases, leads to the formation of EWS::FLI1 [5]. Acting as a transcription factor, the EWS::FLI1 fusion protein alters cellular functions and promotes the expression of key oncogenes such as MYC, ID2, and CCND1, as well as genes associated with super enhancer activities like MEIS1 and APCDD1 [6, 7]. The preferential binding of EWS::FLI1 to GGAA microsatellites creates enhancers that amplify expression of the oncogene, promoting tumor aggression and treatment resistance [8].

However, the structural complexity of EWS::FLI1 and the absence of a defined active site make direct targeting a challenge [9, 10]. Research efforts are thus increasingly focused on understanding and disrupting the regulatory network of EWS::FLI1 as a strategy to counteract its oncogenic effects [2, 6, 11].

The oncogenic potential of EWS::FLI1 is highly dependent on the cellular context, as evidenced by its varying effects across different cell types [12]. In permissive environments like mesenchymal stem (MSC) and neuroectodermal cells, the EWS::FLI1 fusion protein not only drives ES-like transformation and tumorigenesis by promoting cellular proliferation [12], but also significantly contributes to immune evasion [13]. This oncogenic protein modulates immune-related gene expression, alters cytokine profiles to favor immunosuppression, interferes with effective antigen presentation by downregulating MHC class I molecules, and manipulates the tumor microenvironment to encourage the recruitment of immunosuppressive cells like M2 macrophages [13]. EWS::FLI1 toxicity is apparent when expressed in non-permissive cells, as it triggers cellular stress responses that result in cell cycle arrest, apoptosis, or senescence [14].

P300 (E1A Binding Protein P300) and CBP (CREB Binding Protein) are critical histone acetyltransferases that mark promoters and enhancers for gene activation via acetylation of histone H3 at lysine 27 (acH3K27) [15]. This modification is essential for cancer progression in various types such as liver cancer, prostate cancer, melanoma, renal carcinoma, leukemia, lymphoma, and lung cancer [15]. Acetylation by P300/CBP enhances cell proliferation, survival, and metastasis, contributing to therapy resistance and immune evasion mechanisms [15]. There is a crucial partnership in ES, where P300/CBP is dynamically localized to interact with the activation domain of the EWS::FLI1 fusion protein. This results in enhanced oncogenic capacity through chromatin relaxation and remodeling, and subsequent tumor progression [6, 8, 16, 17]. Given the challenges of directly targeting EWS::FLI1, this study focuses on P300/CBP as an alternative therapeutic targets, aiming to disrupt this crucial interaction and thereby reduce oncogenic activity.

Despite their crucial role in cellular processes, the specific function of P300/CBP in ES remains uncharted [15]. This study underscores the dependence of the EWS::FLI1 fusion protein on P300/CBP to govern vital downstream targets crucial for ES cell growth and malignancy. The intriguing regulatory mechanism by which EWS::FLI1 prevents ES cancer cells and permissive cells from entering senescence, acting concurrently as a transcriptional activator of LMNB1 and repressor of P15 (CDKN2B) is unraveled. Thus, the pharmacological targeting of the EWS::FLI1/P300/CBP axis holds promise as a therapeutic approach for ES.

The term “p300/CBP modulator” refers to any molecule that inhibits or degrades p300 and/or CBP (e.g., a p300 and/or CBP inhibitor or a p300 and/or CBP degrader), thereby reducing or disrupting the biological activity and/or function of p300/CBP (protein) or inactivating the biological activity and/or function p300/CBP. In one embodiment the p300/CBP modulator is a p300/CBP inhibitor. In one embodiment the p300/CBP modulator degrades p300/CBP (e.g., a p300/CBP degrader). In one embodiment the p300/CBP modulator is a p300/CBP histone acetyltransferase (HAT) inhibitor. In one embodiment the p300/CBP modulator is a p300/CBP bromodomain inhibitor. In one embodiment the p300/CBP modulator is a compound (e.g., inhibitor) as described in WO 2016/044770. The PCT Application WO 2016/044770 is hereby incorporated by reference in its entirety. In one non-limiting embodiment the p300/CBP modulator is B029-2, XP-524, SGC-CBP30, ISOX-DUAL, A-485, CCS1477, NEO2734, or B026; these compounds are described in documents [19, 68-70], which are hereby incorporated by reference in their entirety. In one embodiment the p300/CBP modulator is an p300 modulator. In one embodiment the p300/CBP modulator is an CBP modulator. In one embodiment the p300/CBP modulator is a p300 inhibitor. In one embodiment the p300/CBP modulator is an CBP inhibitor. In one embodiment the p300/CBP modulator is an p300 degrader. In one embodiment the p300/CBP modulator is an CBP degrader. In one embodiment the p300/CBP modulator is a p300 and CBP modulator. In one embodiment the p300/CBP modulator is a p300 and CBP inhibitor. In one embodiment the p300/CBP modulator is a p300 and CBP degrader. In one embodiment the p300/CBP modulator (inhibitor) is a compound of formula (I) (iP300w):

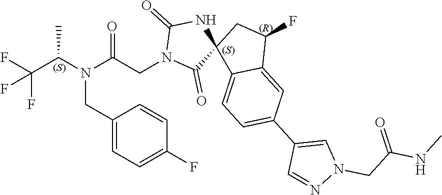

or a pharmaceutically acceptable salt thereof. In one embodiment the p300/CBP modulator (degrader) is a proteolysis targeting chimera (PROTAC). The term “proteolysis targeting chimera” as used herein refers to compounds that facilitate the targeted degradation of proteins. In one embodiment the PROTAC is dCBP-1 or JQAD1. In one embodiment the PROTAC is BT-02C, dCBP-1 or JQAD1 (72).

The term “senotherapeutic”: as used herein, refers to senolytics and senomorphics. Senolytics are agents that selectively eliminate senescent cells (as described below), while senomorphics are agents that suppress or inactivate the senescence-associated secretory phenotype (SASP), the bioactive factors secreted by senescent cells.

The term “senolytic,” as used herein, refers to agents (e.g., pharmaceutical compounds) that selectively target and eliminate senescent cells at various stages of senescence, as defined by the expression of different senescence markers, and facilitate their removal from tissues. In one non-limiting embodiment the senolytic or a pharmaceutically acceptable salt thereof is Dasatinib, Fisetin, Navitoclax, Quercetin, Imatinib, Nilotinib, Bosutinib, Ponatinib, or Asciminib. Sunitinib, Sorafenib, Axitinib, Lenvatinib, Gefitinib, Erlotinib, Vandetanib, Cabozantinib, Pazopanib, Crizotinib, Ceritinib, Brigatinib, Larotrectinib, Entrectinib, Regorafenib, Osimertinib, Alectinib, Trametinib, Neratinib, and Tucatinib, Idelalisib, Alpelisib, Copanlisib, Duvelisib, Buparlisib, Pictilisib, Apitolisib, Dactolisib, Taselisib, Omipalisib, Voxtalisib.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention.

The term “animal” as used herein includes mammals such as humans, higher non-human primates, rodents, domestic, cows, horses, pigs, sheep, dogs and cats. In one embodiment, the animal is a mammal. In one embodiment, the animal is a human.

Pharmaceutically suitable counterions include pharmaceutically suitable cations and pharmaceutically suitable anions that are well known in the art. Examples of pharmaceutically suitable anions include, but are not limited to those described above (e.g. physiologically acceptable anions) including Cl—, Br—, I—, CH₃SO₃—, H₂PO₄—, CF₃SO₃—, p-CH₃C₆H₄SO₃—, citrate, tartrate, phosphate, malate, fumarate, formate, or acetate.

It will be appreciated by those skilled in the art that a compound of the invention comprising a counterion can be converted to a compound of the invention comprising a different counterion. Such a conversion can be accomplished using a variety of well-known techniques and materials including but not limited to ion exchange resins, ion exchange chromatography and selective crystallization.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes. For oral administration, the compounds can be formulated as a solid dosage form with or without an enteric coating.

Thus, the present compounds may be systemically administered, e.g., orally, in combination with a pharmaceutically acceptable vehicle such as an inert diluent, excipient or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 90% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations, particles, and devices.

The active compound may also be administered intravenously or intramuscularly by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina, nanoparticles, and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 1 to about 500 mg/kg, e.g., from about 5 to about 400 mg/kg of body weight per day, such as 1 to about 250 mg per kilogram body weight of the recipient per day.

The compound is conveniently formulated in unit dosage form; for example, containing 5 to 500 mg, 10 to 400 mg, or 5 to 100 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Co-administration of compounds disclosed herein (e.g., p300/CBP (p300 and/or CBP) modulators and senotherapeutics (e.g., senolytics or senomorphics)) generally refers to the simultaneous or sequential administration (e.g., within 1, 6, 12, 24, 36, 48, 72 hours or longer) of the p300/CBP modulator and the senotherapeutic. In one embodiment therapeutically effective amounts of the p300/CBP modulator and the senolytic are both present in the body of the patient. In one embodiment the p300/CBP (p300 and/or CBP) modulator and the senotherapeutic (e.g., senolytic or senomorphic) are administered as a single pharmaceutical compound/formulation (e.g., the two agents are combined in a single dosage form (e.g., tablet, capsule, iv formulation). In one embodiment the p300/CBP (p300 and/or CBP) modulator and the senotherapeutic (e.g., senolytic or senomorphic) are administered essentially simultaneously as separate pharmaceutical compounds/formulations (e.g., the two agents are separate dosage forms (e.g., separate tablets, capsules, iv formulations). In one embodiment the p300/CBP (p300 and/or CBP) modulator and the senotherapeutic (e.g., senolytic or senomorphic) are administered sequentially as separate pharmaceutical compounds/formulations (e.g., the two agents are separate dosage forms (e.g., separate tablets, capsules, iv formulations).

In one embodiment the p300/CBP (p300 and/or CBP) modulator is administered first, followed by the administration (e.g., within 1, 6, 12, 24, 36, 48, 72 hours or longer after administration of the p300/CBP modulator) of the senotherapeutic (e.g., to eliminate senescent cells).

Co-administration of compounds disclosed herein (e.g., p300/CBP modulators and senotherapeutics) with one or more other active therapeutic agents generally refers to simultaneous or sequential administration (e.g., within 1, 6, 12, 24, 36, 48, 72 hours or longer) of the p300/CBP modulator, the senotherapeutic, and the one or more other active therapeutic agent. In one embodiment therapeutically effective amounts of the p300/CBP modulator and the senotherapeutic and the one or more other active therapeutic agent are all present in the body of the patient.

Certain PROTACS and p300/CBP Modulators

### (Compound Names, CAS Numbers, and Structures)

### BT-02C (CAS 3055107-34-8)

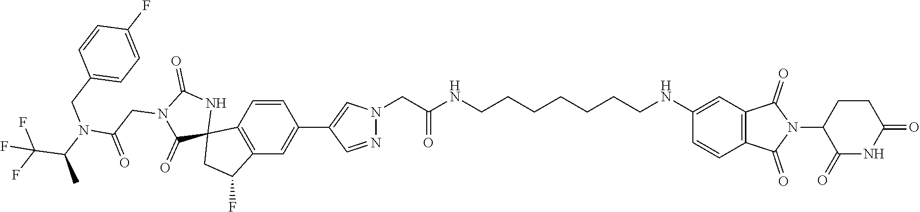 [SMILES: C[C@H](N(Cc1ccc(F)cc1)C(=O)CN1C(=O)N[C@]2(C[C@@H](F)c3cc(-c4cnn(CC(=O)NCCCCCCCNc5ccc6c(c5)C(=O)N(C5CCC(=O)NC5=O)C6=O)c4)ccc32)C1=O)C(F)(F)F]

dCBP-1 (CAS 2484739-25-3)
5H-Pyrazolo[4,3-c]pyridine-5-carboxamide, 3-[7-(difluoromethyl)-3,4-dihydro-6-(1-methyl-1Hpyrazol-4-yl)-1(2H)-quinolinyl]1-[1-[15-[[2-(2,6-dioxo-3-piperidinyl)-2,3-dihydro-1,3-dioxo-1H-isoindol-5-yl]amino]-1-oxo-4,7,10,13-tetraoxapentadec-1-yl]-4-piperidine

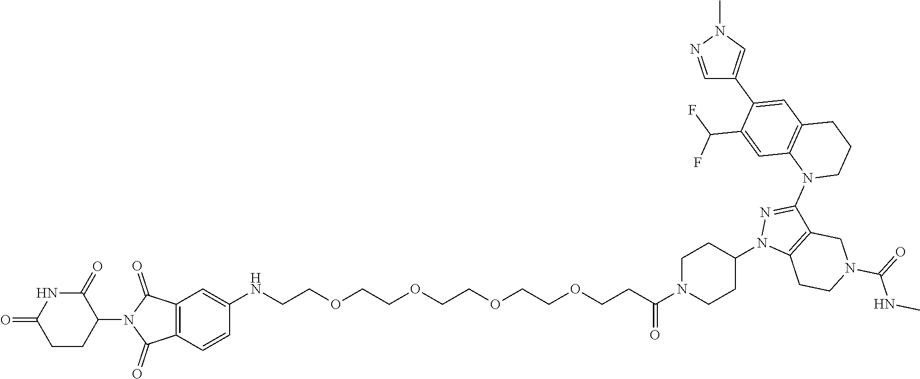 [SMILES: CNC(=O)N1CCc2c(c(N3CCCc4cc(-c5cnn(C)c5)c(C(F)F)cc43)nn2C2CCN(C(=O)CCOCCOCCOCCOCCNc3ccc4c(c3)C(=O)N(C3CCC(=O)NC3=O)C4=O)CC2)C1]

### JQAD1 (CAS 2417097-18-6)

12-((2-(2,6-dioxopiperidin-3-yl)-1,3-dioxoisoindolin-5-yl)amino)-<n-((R)-3-(2-((4-fluorobenzyl)((S)-1,1,1-trifluoropropan-2-yl)amino)-2-oxoethyl)-2′,4′-dioxo-2,3-dihydrospiro[indene-1,5′-oxazolidin]-5-yl)dodecanamide

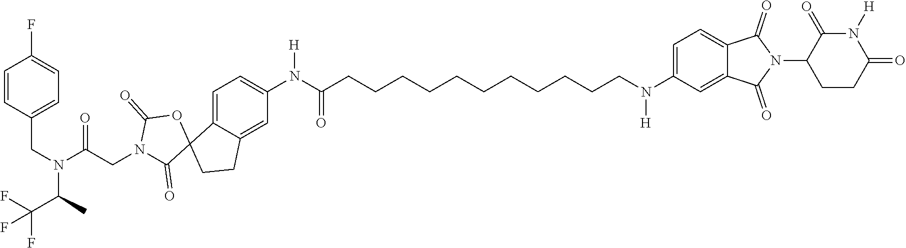 [SMILES: C[C@H](N(Cc1ccc(F)cc1)C(=O)CN1C(=O)OC2(CCc3cc(NC(=O)CCCCCCCCCCCNc4ccc5c(c4)C(=O)N(C4CCC(=O)NC4=O)C5=O)ccc32)C1=O)C(F)(F)F]

### B029-2

1-((R)-3′-(2-((R)-3-cyclopropyl-7-fluoro-2,3-dihydrobenzo[f][1,4]oxazepin-4(5H)-yl)-2-oxoethyl)-4′-methylene-2′-oxo-2,3-dihydrospiro[indene-1,5′-oxazolidin]-5-yl)-3-methylurea

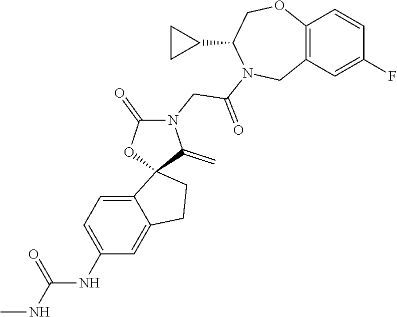 [SMILES: C=C1N(CC(=O)N2Cc3cc(F)ccc3OC[C@H]2C2CC2)C(=O)O[C@@]12CCc1cc(NC(=O)NC)ccc12]

### XP-524 (CAS 2344825-52-9)

N-(1-(1,1-di(pyridin-2-yl)ethyl)-6-(1-methyl-7-oxo-6,7-dihydro-1H-pyrrolo[2,3-c]pyridin-3-yl)-1H-indol-4-yl)ethanesulfonamide

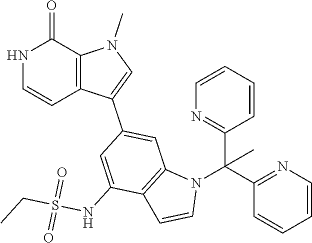 [SMILES: CCS(=O)(=O)Nc1cc(-c2cn(C)c3c(=O)[nH]ccc23)cc2c1ccn2C(C)(c1ccccn1)c1ccccn1]

### SGC-CBP30 (CAS 1613695-14-9)

(S)-4-(1-(2-(3-chloro-4-methoxyphenethyl)-5-(3,5-dimethylisoxazol-4-yl)-1H-benzo[d]imidazol-1-yl)propan-2-yl)morpholine

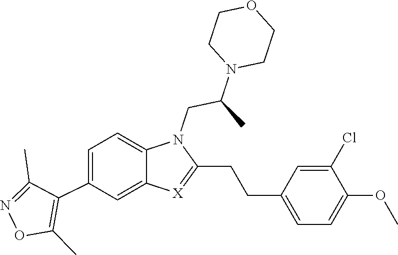

### ISOX-DUAL (CAS 1962928-22-8)

3-[4-[2-[5-(Dimethyl-1,2-oxazol-4-yl)-1-[2-(morpholin-4-yl)ethyl]-1H-1,3-benzodiazol-2-yl]ethyl]phenoxy]propyl]dimethylamine

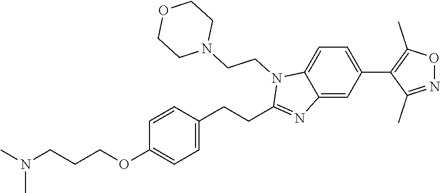 [SMILES: Cc1noc(C)c1-c1ccc2c(c1)nc(CCc1ccc(OCCCN(C)C)cc1)n2CCN1CCOCC1]

### A-485 (CAS 1889279-16-6)

N-(4-fluorobenzyl)-2-((R)-5-(3-methylureido)-2′,4′-dioxo-2,3-dihydrospiro[indene-1,5′-oxazolidin]-3′-yl)-N-((S)-1,1,1-trifluoropropan-2-yl)acetamide

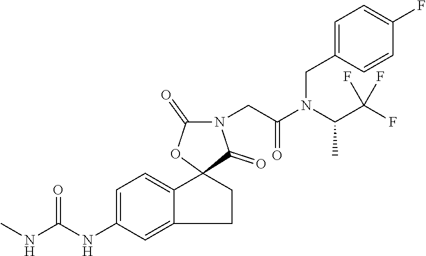 [SMILES: CNC(=O)Nc1ccc2c(c1)CC[C@@]21OC(=O)N(CC(=O)N(Cc2ccc(F)cc2)[C@@H](C)C(F)(F)F)C1=O]

### CCS1477 (CAS 2222941-37-7)

(S)-1-(3,4-difluorophenyl)-6-(5-(3,5-dimethylisoxazol-4-yl)-1-((1r,4S)-4-methoxycyclohexyl)-1H-benzo[d]imidazol-2-yl)piperidin-2-one

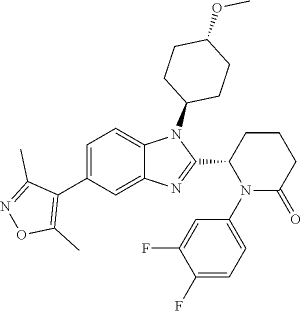 [SMILES: CO[C@H]1CC[C@H](n2c([C@@H]3CCCC(=O)N3c3ccc(F)c(F)c3)nc3cc(-c4c(C)noc4C)ccc32)CC1]

### NEO2734 (CAS 2081072-29-7)

1,3-dimethyl-5-(2-(tetrahydro-2H1-pyran-4-yl)-1-(2-(trifluoromethoxy)ethyl)-1H-benzo[d]imidazol-6-yl)pyridin-2(1H)-one

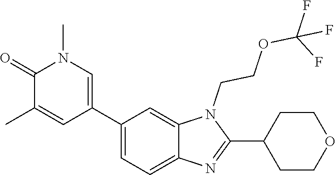 [SMILES: Cc1cc(-c2ccc3nc(C4CCOCC4)n(CCOC(F)(F)F)c3c2)cn(C)c1=O]

### B026 (CAS 2379416-48-3)

(S)-1-(2-((S)-7-fluoro-3-(trifluoromethyl)-2,3-dihydrobenzo[f][1,4]oxazepin-4(5H)-yl)-2-oxoethyl)-5′-(1-methyl-1H-pyrazol-4-yl)-2′,3′-dihydrospiro[imidazolidine-4,1′-indene]-2,5-dione

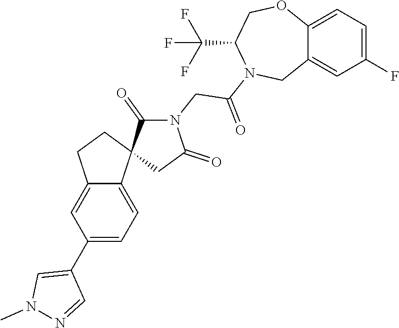 [SMILES: Cn1cc(-c2ccc3c(c2)CC[C@]32CC(=O)N(CC(=O)N3Cc4cc(F)ccc4OC[C@H]3C(F)(F)F)C2=O)cn1]

### FT-7051 (CAS 2304372-79-8)

(1R,3R)-3-((S)-2-((R)-(5-fluoro-2-methoxyphenyl)(hydroxy)methyl)-6-methoxycarbonyl)-7-methyl-6,7,8,9-tetrahydro-3H-imidazo[4,5-f]quinolin-3-yl)cyclohexane-1-carboxylic acid

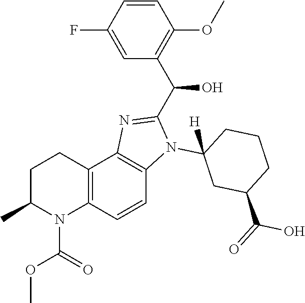 [SMILES: COC(=O)N1c2ccc3c(nc([C@H](O)c4cc(F)ccc4OC)n3[C@@H]3CCC[C@@H](C(=O)O)C3)c2CC[C@@H]1C]

The invention will now be illustrated by the following non-limiting examples.

### Example 1

### Materials and Methods

### Cell Culture and Treatments

All basal media were acquired from HyClone, fetal bovine serum (FBS) was sourced from PeakSerum (Ps-FB3, lot 293Q16), and both Glutamax (Glu) and Penicillin/Streptomycin (P/S) were obtained from GIBCO. The Ewing Sarcoma cell lines TC71, A673, A4573, and SKES1, a generous gift from Dr. Michael Verneris, University of Minnesota, were cultured in DMEM supplemented with 10% FBS, Glu, and P/S. The Ewing Sarcoma cell lines CHLA9, CHLA25, and COG-E-352, a gift from the Childhood Cancer Repository, were cultured in DMEM supplemented with 20% Fetal Bovine Serum, 4 mM L-Glutamine, and 1×ITS (5 μg/mL insulin, 5 μg/mL transferrin, 5 ng/mL selenous acid). The Ewing Sarcoma cell line CADO-ES1, purchased from Cytion (Gaithersburg, US), was cultured in DMEM: Ham's F12 supplemented with 15 mM HEPES. The breast carcinoma (T-47D) cell line was gift from Dr. Reuben Harris, University of Minnesota, rhabdomyosarcoma (A204) cell line was gift from Dr. Dennis Wigle, Mayo Clinic, synovial sarcoma (Fuji), osteosarcoma cell lines U2-OS, 143B, SJSA-1, HOS, and G292, also a gift from Dr. Beau Webber, University of Minnesota, were cultured in DMEM supplemented with 10% FBS, Glu, and P/S. The LHCN-M2 cell line, representing immortalized human myoblasts, was cultured in a proliferation medium composed of F10 supplemented with 20% FBS, 2-mercaptoethanol at a concentration of 1× (GIBCO), 10-9 M dexamethasone (Sigma), 10 ng/mL bFGF (Peprotech), along with Glu/P/S [32]. All cell cultures were maintained at 37° C. in a 5% CO2 atmosphere.

### Cell Viability (ATP) Assay and Synergy Calculation

Cell lines were plated in 96-well plates at a density of 1×105 cells per well. The following day, iP300w or its stereoisomers were administered as part of the therapeutic regimen. Cell viability was assessed using the CellTiter-Glo® Luminescent Cell Viability Assay kit (Promega), adhering closely to the manufacturer's guidelines. Luminescence measurements, indicative of cell viability, were taken using the POLARstar Optima Microplate Reader (BMG Labtech, Offenburg, Germany).

Senolytic agents Dasatinib (SML2589, Sigma-Aldrich), Quercetin (73932, STEMCELL Technologies), Fisetin (S2298, Selleck Chemicals), and Navitoclax (NC1731582, Fisher Scientific) were diluted with DMSO and medium to concentrations of 0.01 μM, 0.1 μM, 1 μM, μM, 20 μM, and 50 μM. In the control group, DMSO concentration was adjusted to match the highest treatment concentration of 50 μM. Both senolytics and iP300w were applied either alone or in combination for a duration of 72 hours, followed by ATP assay analysis as previously described.

Following the completion of the ATP viability assays, data were imported into SynergyFinder Plus for synergy score analysis. Using the Highest Single Agent (HSA) model, synergy scores across the tested conditions were calculated, focusing on the combined effects of iP300w (0 μM, 1 μM) and senolytics (Dasatinib, Quercetin, Fisetin, and Navitoclax) across a range of concentrations (0 μM, 0.01 μM, 0.1 μM, 1 μM, 10 μM, 20 μM, and 50 μM). The analysis was performed using the default settings of the HSA model, which compares the efficacy of combination treatments relative to the most effective single-agent treatment in each condition.

### Antibodies, Western Blot, and Immunofluorescence

For western blot analyses, cell lysates were prepared using RIPA buffer enhanced with a protease inhibitor cocktail (Complete, Roche). Proteins were resolved by electrophoresis on 10% SDS-PAGE gels and subsequently transferred to PVDF membranes. The membranes were incubated with primary antibodies diluted in 5% skim milk in TBST either overnight at 4° C. or for 1 hour at room temperature (RT). Following primary antibody incubation, membranes were treated with an appropriate RP-conjugated secondary antibody for 1 hour at RT. After washing with TBST, the protein bands were visualized using Pierce ECL western blotting substrate (Thermo Scientific).

For immunofluorescence assays, cells grown in 96-well plates were fixed with 4% paraformaldehyde (PFA) for 10 minutes, washed twice with PBS, and then permeabilized with 0.3% Triton X-100 for 30 minutes. Blocking was performed with 3% BSA for 1 hour at room temperature. Primary antibodies were diluted in 3% BSA and incubated overnight at 4° C. Subsequently, cells were incubated with a suitable fluorophore-conjugated secondary antibody for 60 minutes at room temperature. Nuclei staining was achieved using DAPI (dilution 1:5000, Sigma).

The antibodies utilized in this study included GAPDH-RP (1:5000, Proteintech 60004), rabbit anti-Histone H3K18Ac (1:500, Abcam ab1191), rabbit anti-Histone H3K27Ac (1:500, Abcam ab1791, lot: GR3297878-1), rabbit anti-LMNB1 (1:1000, Abcam ab16048), rabbit anti-γH2AX (1:1000, Cell Signaling 9718s), rabbit anti-Ki-67 (dilution 1:250, Cell Signaling 9129T), anti-mouse P300 (1:500, Active Motif 61401), rabbit anti-CBP (1:1000, Cell Signaling 7389S), secondary Alexa Fluor 555 Goat Anti-Rabbit (1:500, Invitrogen), HRP-conjugated anti-rabbit (1:5000, Jackson Immuno Research 111-035-003, lot: 149393), and RP-conjugated anti-mouse (1:2500, Novus NBP1-75130, lot 58-173-090418).

In Vivo Mouse Tumor Formation and iP300w Evaluation

Mouse tumor formation and evaluation of iP300w in vivo were carried out at the University of Minnesota Research Animal Resources facility, adhering to protocol (2209-40422A) approved by IACUC. Mice were grouped by matching sex and age, with random assignments to control or experimental groups. Immunodeficient NSG mice, three months old, were transplanted with 0.56×107 SKES1 cells in 100 μL of medium and Matrigel (Corning) mix. iP300w was prepared in DMSO (10 mM), diluted in 100 μL PBS, and administered intraperitoneally twice daily, with the control group receiving the vehicle only. Treatment was initiated one day after transplantation. Tumor size was measured every 3 days, using the formula 0.5×length×width2. Mice were identified by number, and the investigator was blinded to the treatment status during tumor dissection, weighing, and photographing.

### RNA Isolation, Quantitative Real-Time RT-PCR, and RNA Sequencing

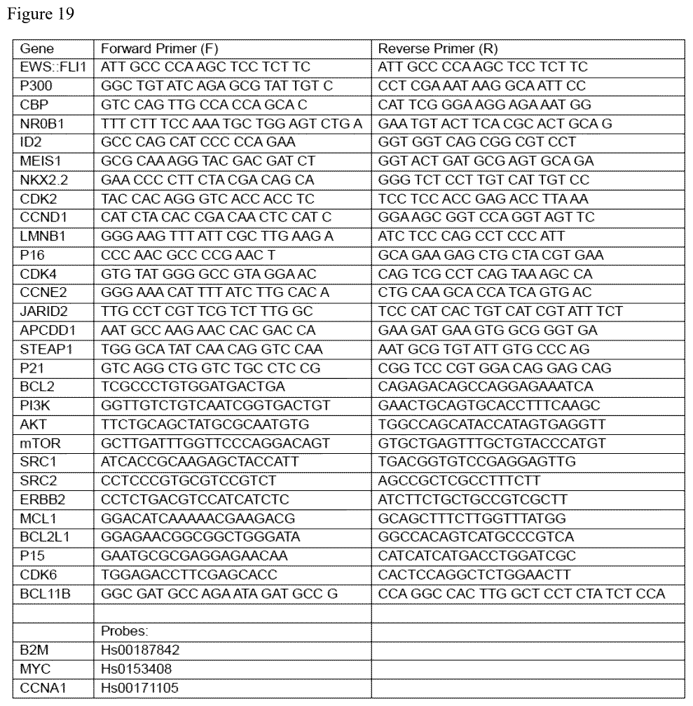

RNA was isolated employing the Zymo RNA extraction kit, and complementary DNA (cDNA) was synthesized from 0.5 μg of total RNA using an oligo-dT primer and the Verso cDNA Synthesis Kit from Thermo Scientific, adhering strictly to the provided protocol. Quantitative PCR (qPCR) analyses utilized Premix Ex Taq (Probe qPCR, Takara) or SYBR Green for detection. Gene expression quantification was anchored to GAPDH or B2M reference genes and computed using the 7500 System Software through the ACT method by Applied Biosystems. Primers and probes are listed in Table 1 (FIG. 19). For RNA sequencing (RNA-seq), libraries were prepared from 500 ng of total RNA extracted from cells treated with iP300w (1.01 μM) for 4 hours. Library preparation and sequencing (2×150 bp, 20M paired-end reads) was done at Azenta Life Science, USA.

### RNA Interference

SKES1, A4573 and TC71 cells were plated in 96-well plates at a density of 5×104 cells per well for assessing cell viability, or in 24-well plates at 1.5×105 cells per well for RNA isolation purposes. On the following day, cells were transfected with 50 nM siRNA targeting human P300 (L-003486-00-0005) and CREBBP (L-003477-00-0005), or a non-targeting scrambled control siRNA (SMARTpool, Dharmacon), employing Lipofectamine RNAiMAX (Invitrogen) as the transfection agent. RNA was extracted 48 hours after transfection, and the impact on cell viability was evaluated at both 48 and 72 hours post-transfection. Similarly, SKES1 cells were transfected with 50 nM siRNA targeting human P15 (L-003245-00-0005) under the same conditions. Downstream analyses were conducted after 24 hours post-transfection and following 48 hours of treatment with iP300w.

### Retrovirus Infection

LMNB1 (GFP-LMNB1) and control GFP were overexpressed in SKES1 cells using retrovirus vectors pQCXIP-GFP and pQCXIP-GFP-myc-HsLMNB1, generously provided by Dr. Stephen A. Adam from Northwestern University [60]. The retroviruses were produced in 293T packaging cells using pVSV-G (Clontech). Supernatant collected at 48 and 72 hours post-infection, mixed with polybrene (10 μg/ml), was used to infect SKES1 cells. Selection was achieved by puromycin (2 μg/ml), and confirmation was conducted by FACS analysis of GFP-positive cells.

### β-Galactosidase (β-Gal) Cell Staining

Cells were seeded in 96-well plates and at 50% confluence. The medium was removed, and cells were rinsed with PBS and fixed using 1× fixative from the senescence β-galactosidase staining kit (9860, Cell Signaling Technology) for 15 minutes. After additional PBS washes, each well was stained with 50 μL of β-gal staining solution and incubated at 37° C. overnight. For senescence delay experiments, the staining period was shortened to 4 hours for non-treated groups showing significant β-Gal positivity. Senescent cells were identified by β-galactosidase staining and quantified in at least three random fields.

### Annexin V Staining

Apoptosis was assessed using the APC Annexin V Apoptosis Detection Kit with 7-AAD (Biolegend, 640930), strictly following the manufacturer's guidelines. After treatment, cells were harvested and labeled according to the kit's protocol. Analysis was performed on a BD FACSCanto II flow cytometer (Becton Dickinson, US), focusing on capturing mean fluorescence intensity (MFI) data. This data was then processed using FlowJo software (Tree Star, USA) to quantify apoptotic events.

### Bioinformatics

Paired-end Illumina sequencing reads were processed using TrimGalore (version 0.6.0), and transcript quantification was performed with Salmon (version 1.2.1) utilizing human Gencode annotations (version 34) and applying the GC-bias correction feature. Data importation into R (version 4.0.2) was achieved using tximeta (version 1.6.3). Differential expression analysis was conducted using DESeq2 (version 1.28.1). Visualization tools employed included ComplexHeatmap (version 2.4.3), clusterProfiler (version 3.16.1), UpsetR (version 1.4), and ggplot2 (version 3.3.2). Pathway enrichment analysis was performed using KEGG (www.genome.jp/kegg/), with figures generated by clusterProfiler (version 3.16.1). Clustering of patients was executed using NMF (version 0.27). Survival analysis utilized the Survival package (version 3.5) for single Cox models and glmnet (version 4.1) for Lasso Cox models. Lasso Cox regression involved 10-fold cross-validation to identify the optimal value for the regularization parameter lambda (k) [71], selected to minimize the partial likelihood deviance. The coefficient profiles of candidate genes were analyzed as a function of log 2(lambda), with key predictors retained at the optimal λ value.

ChIP-seq data were analyzed using IGV (version 2.16.2).

### Statistics

Statistical analyses were conducted using Graphpad Prism software, except as noted. The sample sizes were determined based on previous experience with similar assays to achieve sufficient statistical power. Variance within each group was comparable. Group comparisons were made using either one-way or two-way analysis of variance (ANOVA), with subsequent Tukey's post-hoc tests for specific comparisons. Statistical significance was established at p-values less than or equal to 0.05.

### Results

siRNA-Mediated Knockdown of P300/CBP Affects ES Cell Viability

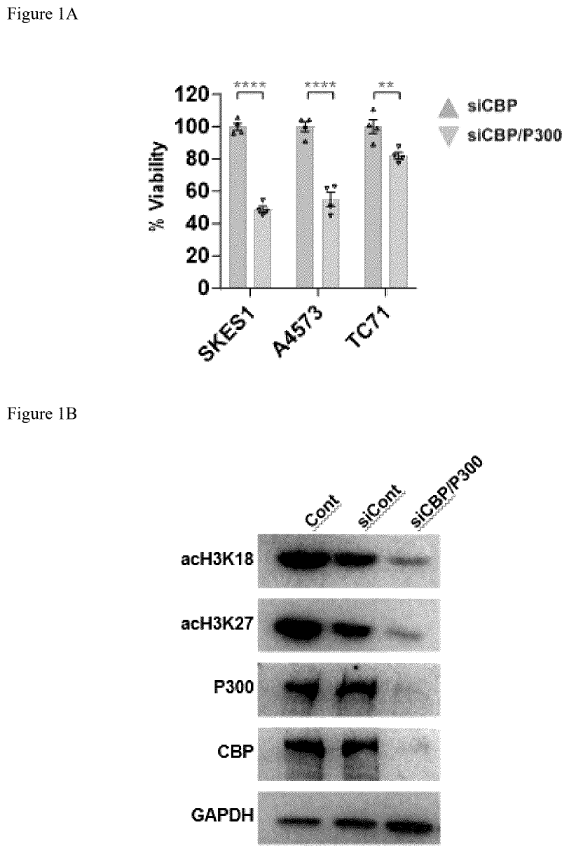

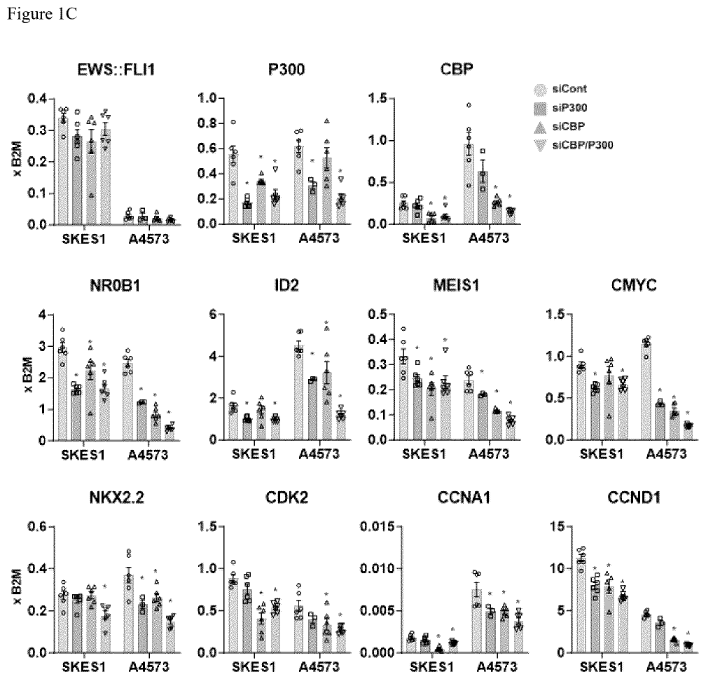

While P300/CBP is recognized as a promoter of cancer cell proliferation [15, 18], its impact on ES viability remains uncertain. The influence of P300/CBP on the survival of three ES cell lines, SKES1, A4573, and TC71, was evaluated through siRNA-mediated knockdown. P300/CBP-targeted siRNA decreased cell viability across all examined ES cell lines, underscoring the vital role these histone acetyltransferases play in cell survival (FIG. 1A). The efficiency of siRNA-mediated knockdown was confirmed by assessing P300 and CBP protein levels via Western blot (FIG. 1i). Knockdown of P300/CBP led to a reduction in acetylated H3K18 and H3K27, indicating a shift toward a transcriptionally repressive chromatin state. Knockdown of either P300 or CBP reduced the expression of well-known targets of EWS::FLI1, such as NR0B1, MEIS1, and C-MYC levels in SKES1 and A4573 ES cell lines. However, silencing both P300 and CBP together had a more pronounced effect, resulting in heightened downregulation across a plethora of targets (FIG. 1C). Thus, knockdown of P300/CBP by RNAi emphasizes its indispensable role within the EWS::FLI1 transcriptional axis and in maintaining ES cell viability.

### Pharmacological Inhibition P300/CBP Inactivates EWS::FLI1 Oncogenic Activity

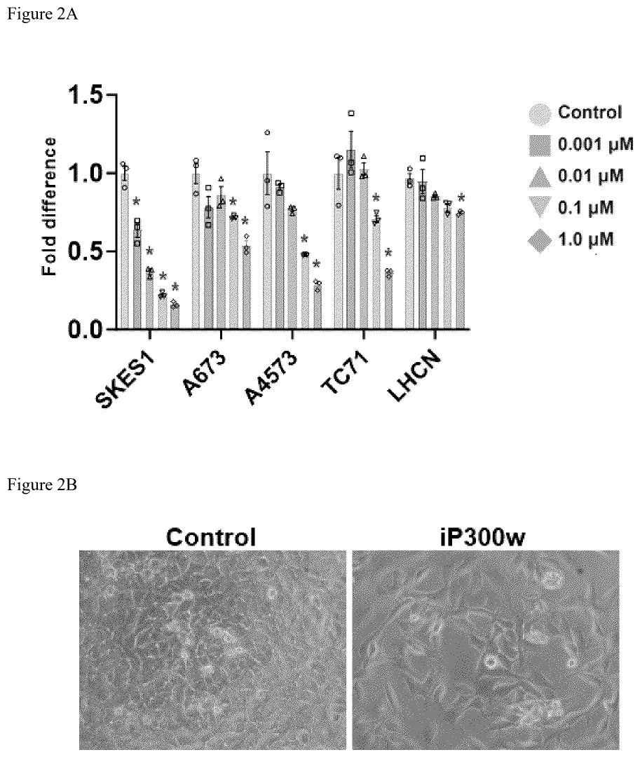

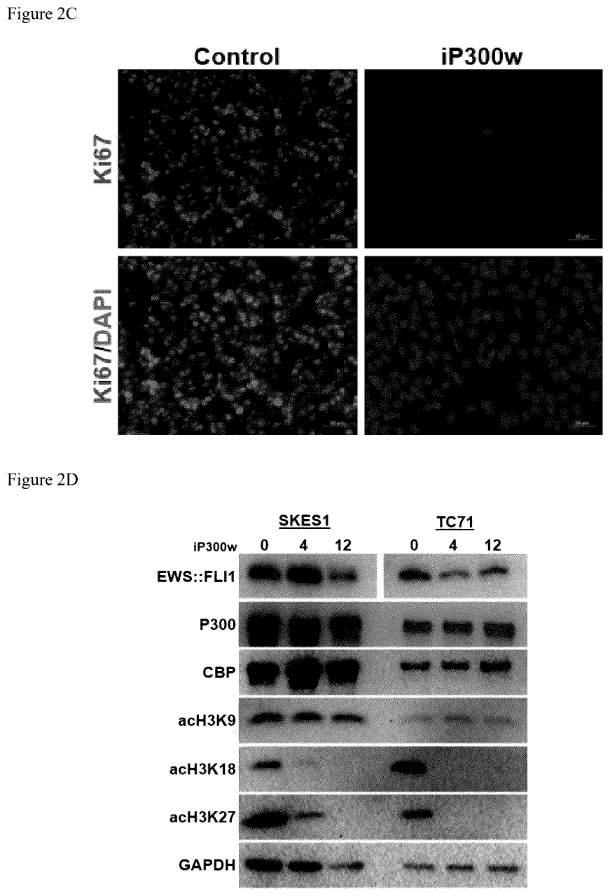

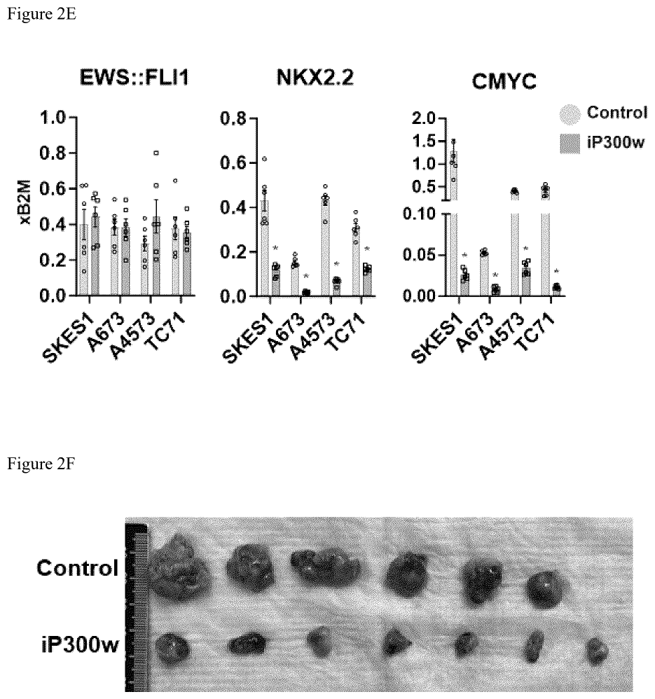

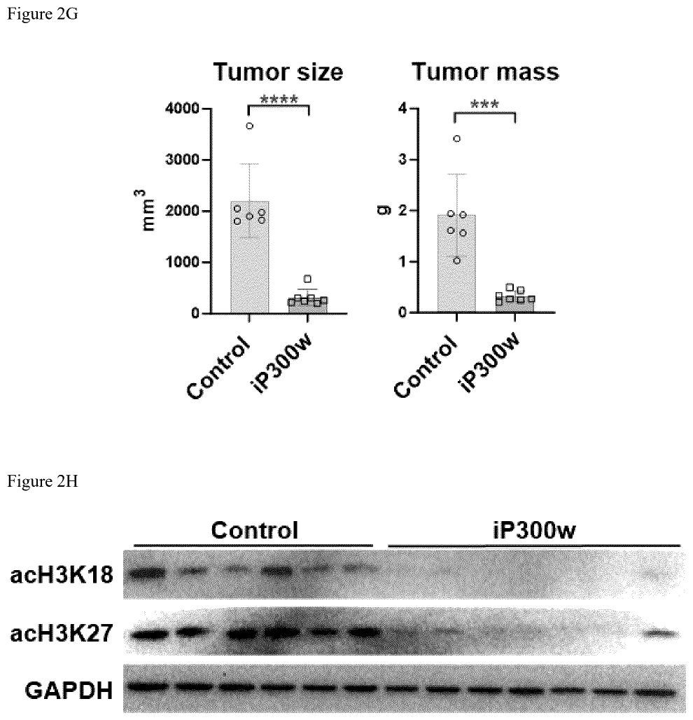

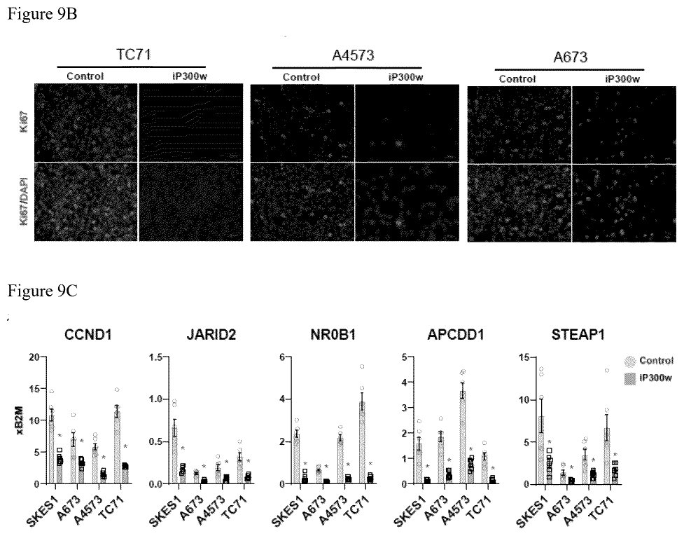

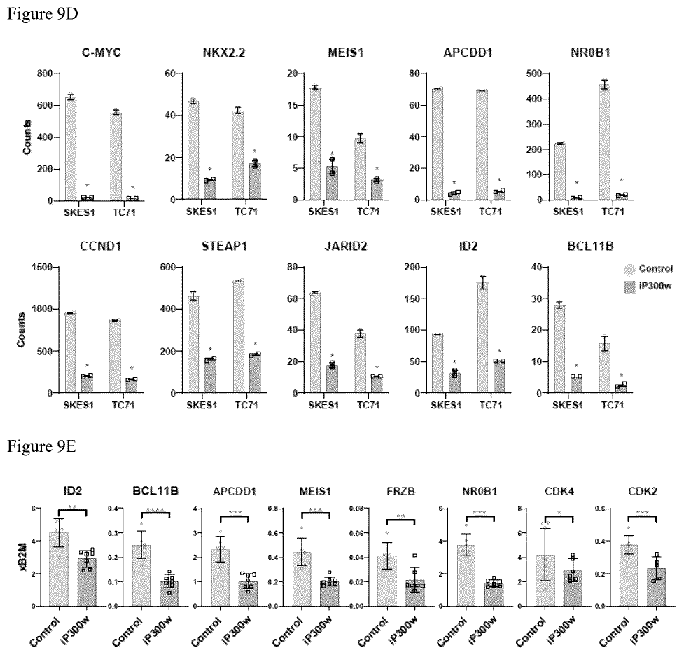

To pharmacologically emulate the effects of P300/CBP siRNA-mediated knockdown, ES cell lines were treated with the recently developed P300/CBP inhibitor, iP300w [18]. The treatment reduced cell viability in a dose-dependent manner across all tested ES cell lines. All tested ES cell lines exhibited significant sensitivity to iP300w at a concentration of 0.1 μM after 48 hours of treatment (FIG. 2A and FIG. 9A). In contrast, the viability of the non-sarcoma human myoblast LHCN cell line and the osteosarcoma cell line remained unaffected at this concentration (FIG. 2A and FIG. 9A). However, a slight but statistically significant decrease in cell viability was observed in the LHCN and SJSA-1 cell lines at 1.01 μM. The impact of P300/CBP inhibition on cell proliferation and morphology was readily observable under bright-field microscopy. iP300w-treated samples exhibited a marked decrease in cell confluence, along with a noticeable increase in cell size and a more flattened, spread-out morphology. (FIG. 2B). Ki-67 staining demonstrated almost complete suppression of cell proliferation at 48 hours of the iP300w treatment (FIG. 2C and FIG. 9B).

Next, the transcriptional and epigenetic effects of iP300w treatment in both SKES1 and TC71 cell lines was investigated. Within 4 hours, acetylation at H3K18 and H3K27 notably decreased, and was entirely diminished by the 12-hour mark (FIG. 2D). As anticipated, there was no change in acetylation levels on H3K9, affirming the iP300w specific inhibition of P300/CBP-mediated H3 acetylation [19]. Concurrently, protein levels of P300 and CBP remained stable, suggesting iP300w modulates the activity of these acetyltransferases without altering their expression (FIG. 2D). Moreover, RT-qPCR analysis at 24 hours post-iP300w treatment revealed a significant downregulation of EWS::FLI1 target genes, notably NKX2.2, NR0B1 and C-MYC (FIG. 2E, FIG. 9C). Remarkably, the sensitivity of EWS::FLI1 transcriptional activity to treatment was evident within just 4 hours (FIG. 9D). Notably, EWS::FLI1 protein levels decreased with iP300w treatment while transcription remained unaffected. This suggests that the stability of EWS::FLI1 may be dependent on P300/CBP-mediated acetylation (FIG. 2D, E), similar to the relationship between P300/CBP and other transcription factors like TP53, SOX10, and MCL-1 [20-22].

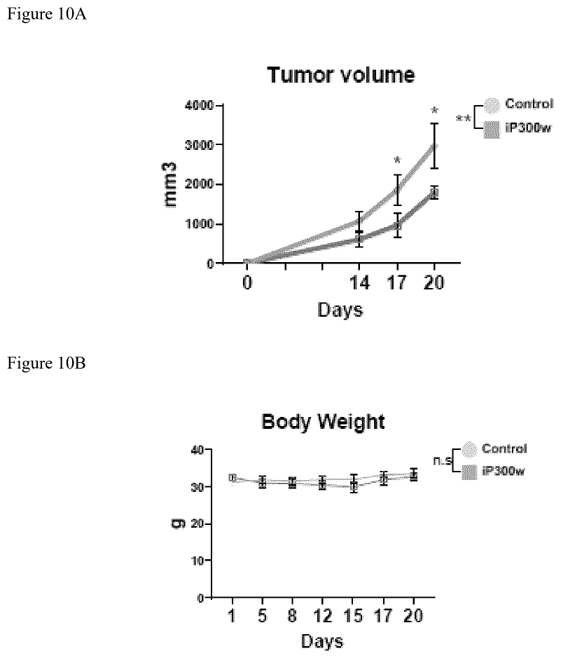

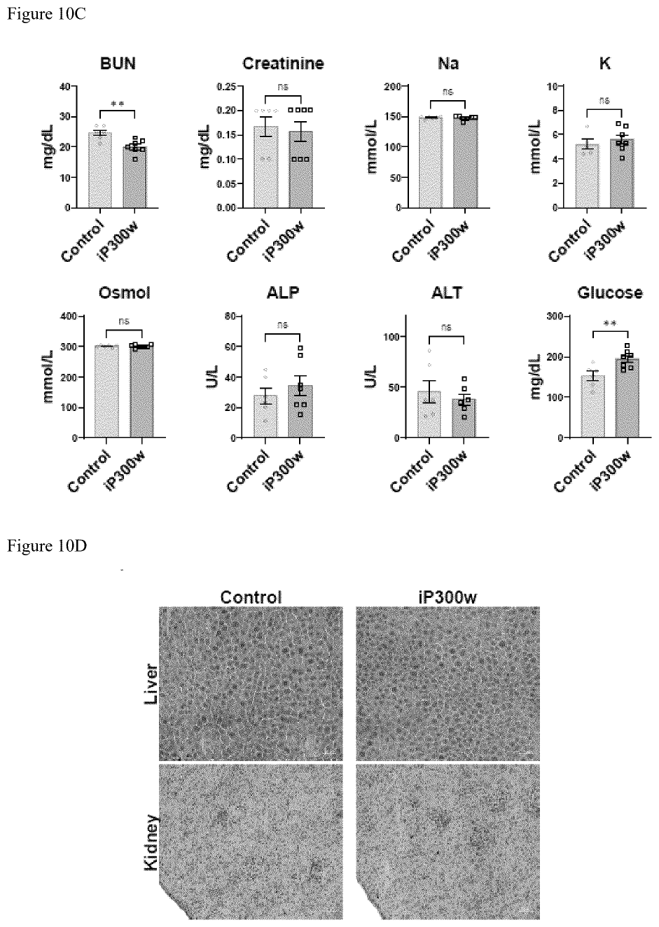

Finally, the efficacy of iP300w in suppressing ES tumor formation in a SKES1 xenograft tumor model (FIG. 2F, G and FIG. 10A) was assessed. Mice treated with iP300w exhibited significant reductions in both tumor size and mass compared to control groups. By the end of the experiment (day 14), average tumor volumes were significantly smaller in the treated group (300 mm³, 0.3 g) compared to the untreated control group of mice (2200 mm³, 2 g) (FIG. 2H, G). Consistent with the in vitro observations, treated tumors showed a notable decrease in H3K18 and H3K27 acetylation, as well as in the expression of EWS::FLI1 target genes (FIGS. 2H and I, and FIG. 9E). To evaluate the potential side effects of iP300w treatment, the body weight changes (FIG. 10B) and assessed serum levels of BUN, creatinine, sodium, potassium, osmolality, ALP, ALT, and glucose (FIG. 10C) was monitored. Among all the parameters, only glucose showed a slight upregulation in the iP300w-treated group. BUN was actually modestly decreased with statistical significance in the treated group. Furthermore, histopathological analysis revealed no noticeable tissue damage or pathological changes in the liver and kidneys (FIG. 10D). In summary, iP300w efficiently suppresses EWS::FLI1/P300/CBP oncogenic axis both in vitro and in vivo.

### EWS::FLI1 Knockdown and P300/CBP Inhibition Induce Similar Transcriptional Alterations in ES

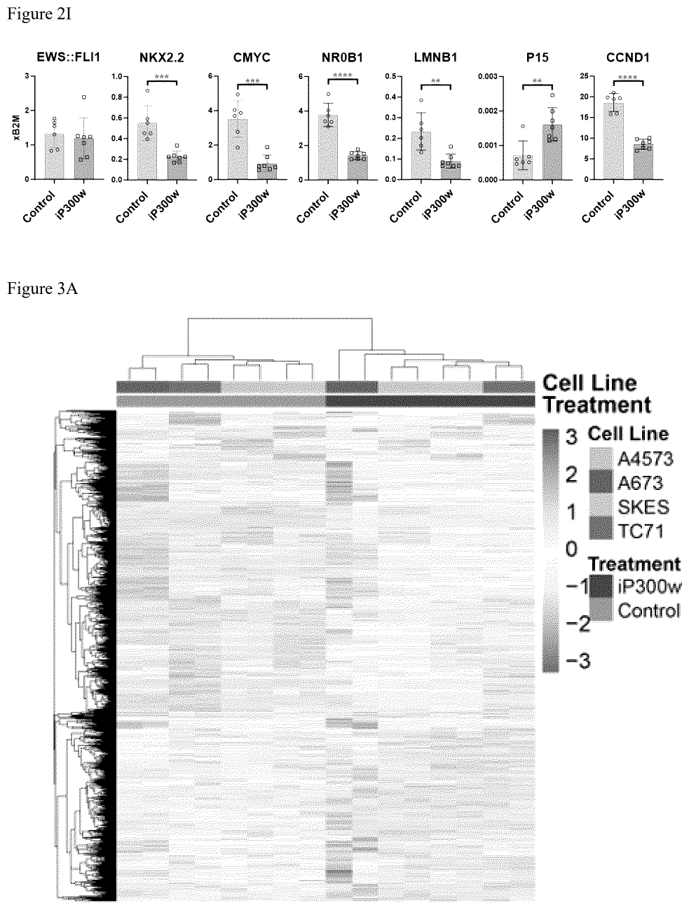

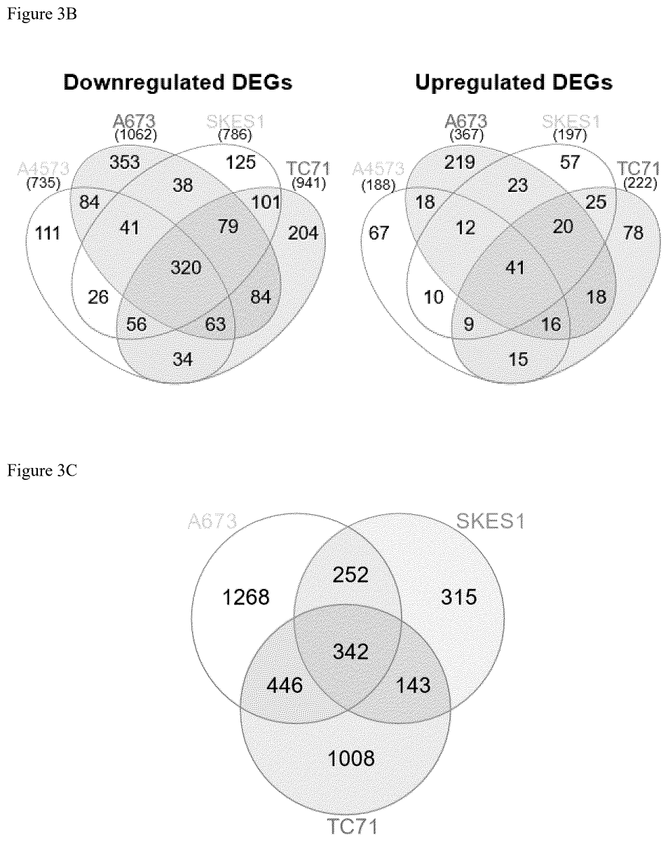

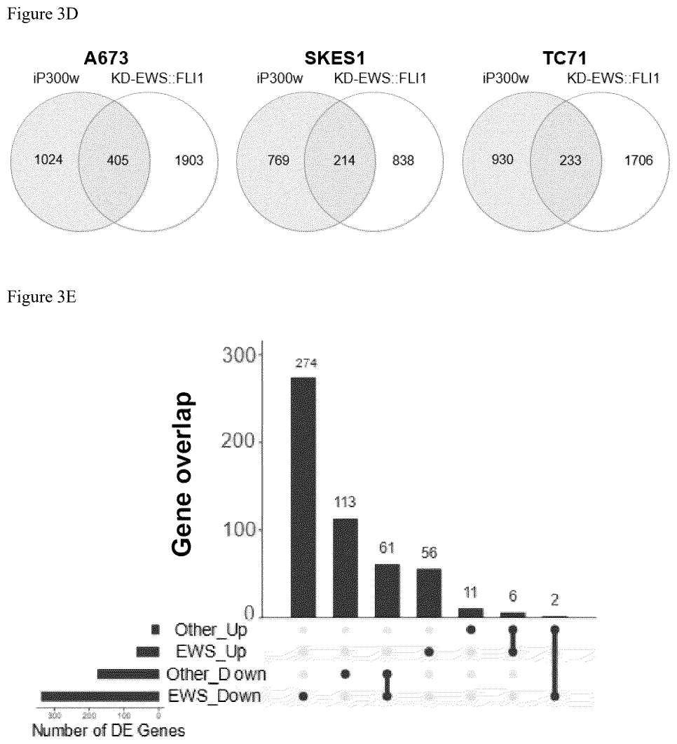

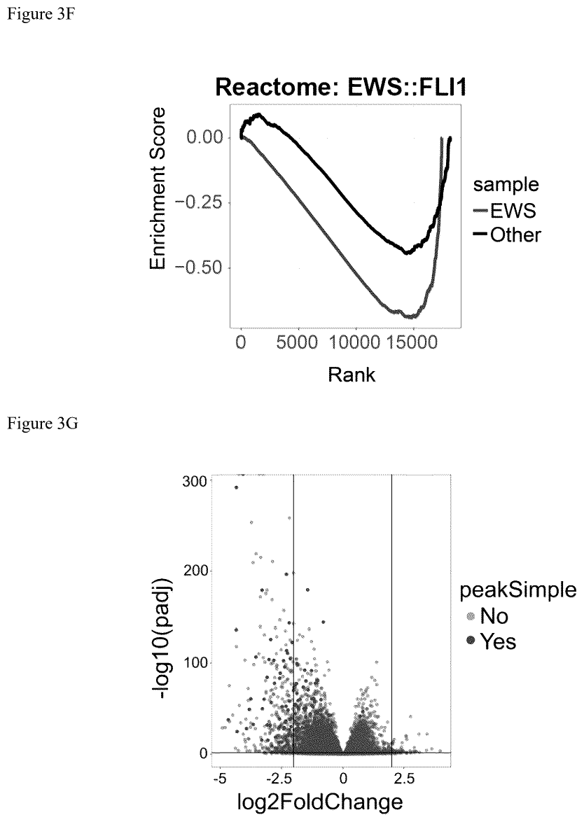

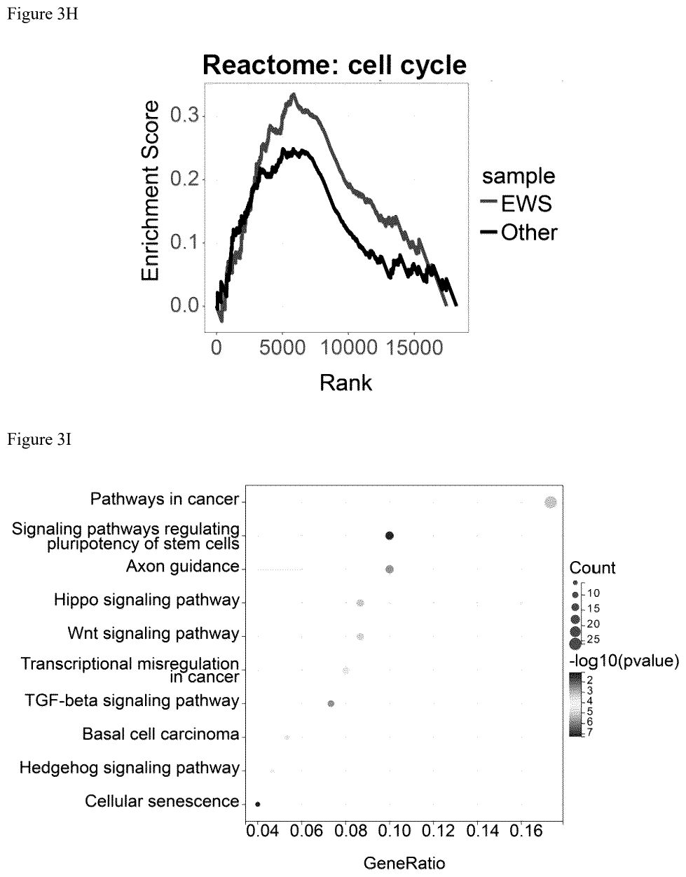

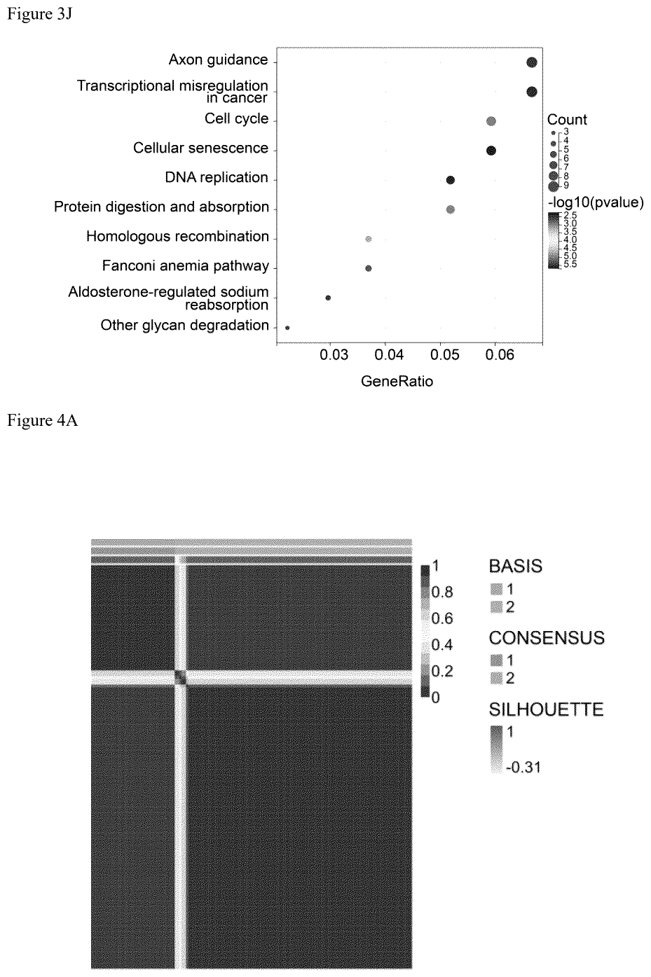

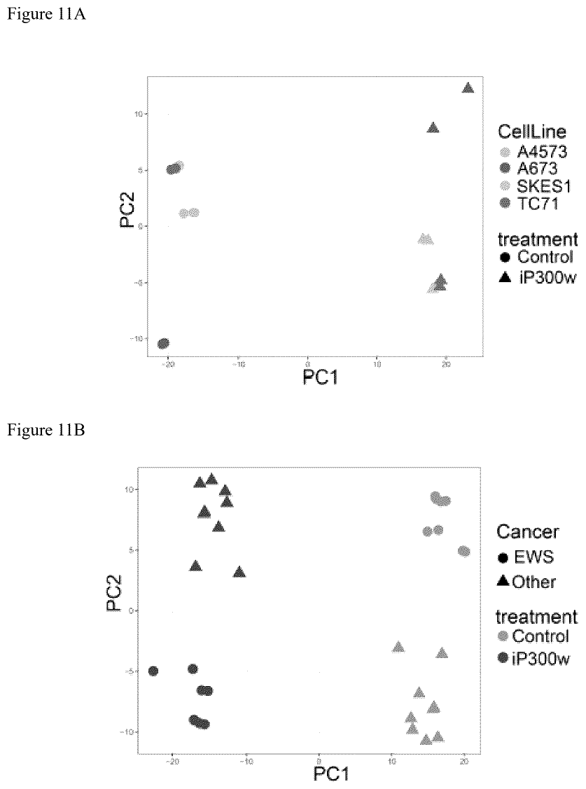

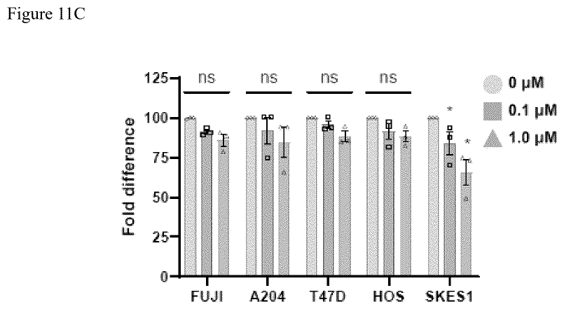

Global transcriptional analysis was employed in iP300w-treated ES cells to identify molecular mechanisms underlying EWS:FLI1/P300/CBP-driven malignancy. An advantage of chemical inhibition over siRNA knockdown is its rapid effect. To capture immediate and highly sensitive transcriptional responses while avoiding secondary targets, RNA-Seq was performed at 4 hours post-treatment. Comparison revealed distinct clustering based on differentially expressed genes (DEGs), effectively segregating treated cells from controls (FIG. 3A). Principal Component Analysis (PCA) revealed a clear trend: while different ES cell lines clustered together in the control set, they were distinctly separated in the iP300w-treated groups, highlighting the substantial impact of iP300w treatment on cell transcriptomes (FIG. 11A and FIG. 11B). Differential gene expression (DGE) analyses revealed a pronounced predominance of downregulated genes over upregulated ones, in a ratio of 4:1, indicating widespread transcriptional repression induced by the treatment (FIG. 3B). This trend remained consistent across all four ES cell lines, demonstrating a notable overlap, especially among the downregulated genes.

To assess the dependency of EWS::FLI1 transcriptional activity on P300/CBP, transcriptional alterations following EWS::FLI1 knockdown (48 hours) and P300/CBP inhibition (4 hours) was compared. Transcriptional profiles of A673, TC71, and SKES1 after EWS::FLI1 knockdown were accessed through the Gene Expression Omnibus (GEO) data repository [23]. Although the knockdown approach requires a much longer timeframe, meaning much greater involvement of indirect targets, Venn diagram analysis indicated a substantial number of common genes affected by EWS::FLI1 knockdown across different ES cell lines, similar to what was observed previously with P300/CBP inhibition (FIGS. 3B and C). Subsequent comparison between the two approaches revealed significant overlaps of approximately one-fourth of the differentially expressed genes (DEGs), indicating a shared transcriptional response to both EWS-FLI1 knockdown and iP300w treatment (FIG. 3D).

To underscore the significance of P300/CBP in EWS::FLI1 activity, UpsetR for a comparative analysis of RNA-Seq data between ES cell lines and non-ES cell lines treated with iP300w (FIG. 3E) was utilized. In the non-ES cell line pool, diverse cancer cell lines such as breast carcinoma (T-47D), rhabdomyosarcoma (A204), synovial sarcoma (Fuji), and osteosarcoma (HOS, U2-OS) were included. The comparison of DEGs revealed that ES cells exhibited heightened sensitivity to iP300w as compared to other cancer cell lines. For instance, 330 DEGs were uniquely affected in ES cells compared to 124 affected genes in other lines (FIG. 3E). Furthermore, Gene Set Enrichment Analysis (GSEA) demonstrated a robust transcriptional correlation between iP300w-treated ES cells and mesenchymal stem cells (MSC) overexpressing EWS::FLI1 (p=8.54e-52; Reactome EWS::FLI1) (FIG. 3F). Notably, 74 of DEGs influenced by the treatment contained EWS::FLI1 binding sites within 50 KB of the promoter region (FIG. 3G). Additionally, GSEA revealed a more pronounced impact on the cell cycle in iP300w-treated ES cell lines compared to other cancer cell lines (p=2.18e-4; Reactome: cell cycle; FIG. 3H). This is further supported by the ATP assay results, which showed that iP300w did not significantly affect the viability of non-ES cell lines (FIG. 9A and FIG. 11C).

Finally, KEGG pathway analysis revealed modulation of pathways associated with cancer, stem cell pluripotency, and cellular signaling in iP300w-treated cells. Particularly notable changes were observed in the Hippo, Wnt, and Hedgehog pathways, indicating the widespread effects of P300/CBP inhibition on regulatory networks crucial for tumorigenesis and cell differentiation (FIG. 3I). Conversely, in EWS::FLI1 knockdown cells, DNA replication and cell cycle-related pathways were predominantly affected (FIG. 3J). Among the common most affected pathways between iP300w treatment and EWS::FLI1 knockdown were “Transcriptional misregulation in cancer” and “Cellular senescence”. Collectively, these findings highlight that at the transcriptional level, inhibition of P300/CBP with iP300w can yield effects comparable to EWS::FLI1 knockdown in ES cells.

### Differential Survival Outcomes in ES Patients Linked to P300/CBP Related Genes

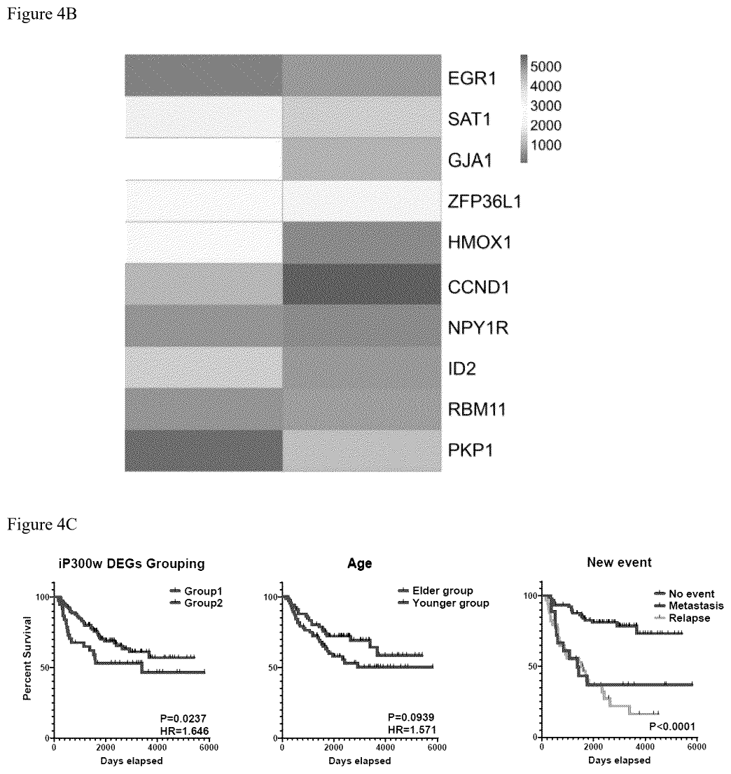

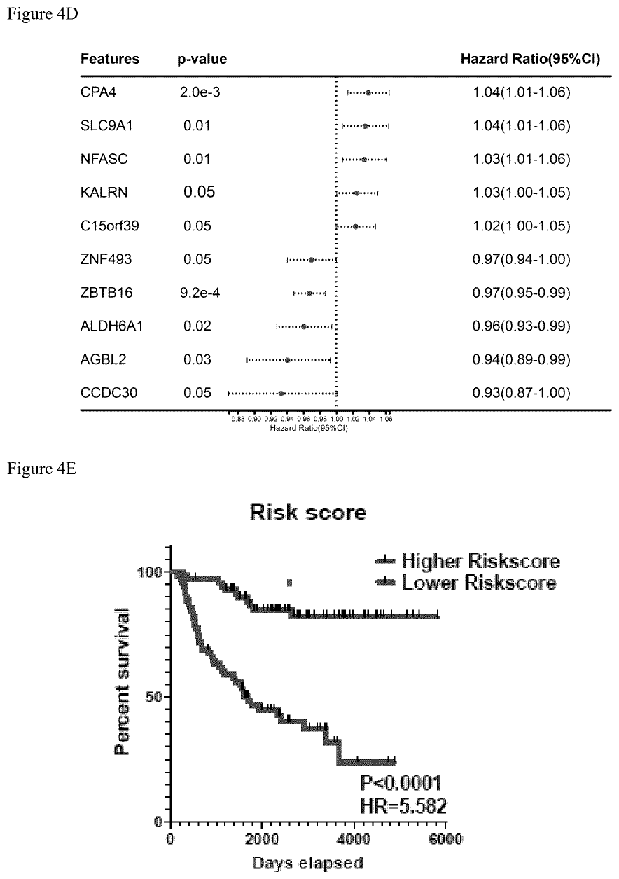

Expanding upon the gene expression findings, the potential clinical relevance of EWS::FLI1/P300/CBP axis-related genes was explored. The DEGs from iP300w-treated ES cell lines with the overall gene expression profiles of biopsy samples from 142 Ewing Sarcoma patients archived in the International Cancer Genome Consortium (ICGC) database and GSE63157 [24] were correlated. Employing Non-negative Matrix Factorization (NMF), these patients were categorized into two distinct groups based on the expression patterns of the iP300w affected DEGs. This stratification is visually represented in an NMF clustering consensus map (FIG. 4A), while a heatmap (FIG. 4B) further elaborates on the expression profiles of the top five genes from each group, highlighting the potential impact of these DEGs on patient outcomes.

To explore the clinical implications of these findings, survival analysis of the two distinct patient groups identified via NMF clustering was conducted. There were significant differences (p=0.0237) in survival outcomes among these groups (FIG. 4C), underscoring the potential prognostic relevance of P300/CBP related genes in ES. The patient samples by median age were stratified; however, this categorization did not reveal a significant impact on survival (p=0.0939). Additionally, the role of metastatic and relapse state in patient prognosis. Was assessed. The analysis revealed that event-fee patients had significantly better survival compared to those with metastasis (Hazard Ratio=0.4861, p=0.0014) and those with relapse (Hazard Ratio=0.4608, p=0.0124). However, there was no significant difference in survival between the groups with metastasis and relapse (p=0.913) (FIG. 4C). The overall survival analysis across the three groups was highly significant (p<0.001).

Given the classification of samples into two categories through NMF, a direct understanding of the relationship between DEGs and patient overall survival was not immediately clear. To bridge this gap, Cox regression analysis to examine the link between DEG expression and overall survival was employed, identifying 74 genes significantly associated with patient survival (Wald p<0.05), with the top 5 and bottom 5 hazard ratio genes presented for clarity (FIG. 4D). Recognizing patient survival is unlikely to be regulated by a single gene, a Lasso Cox analysis of these genes to construct a model correlating gene expression with overall survival (FIG. 12) was conducted. The model assigned scores based on the expression of different genes, and higher scores were identified as a significant risk factor, evidenced by a Hazard ratio of 5.582 and p<0.0001, indicating a strong association with patient survival, comparable to the impact of metastasis or relapse (FIG. 4E).

This clinical analysis, based on survival data, illuminates the pivotal prognostic role of P300/CBP related genes in ES, revealing a robust correlation between patient stratification according to distinct gene expression patterns and diverse overall survival outcomes. These findings underscore the compelling prognostic relevance of targeting P300/CBP in ES.

### P300/CBP Inhibition Triggers Senescence in ES

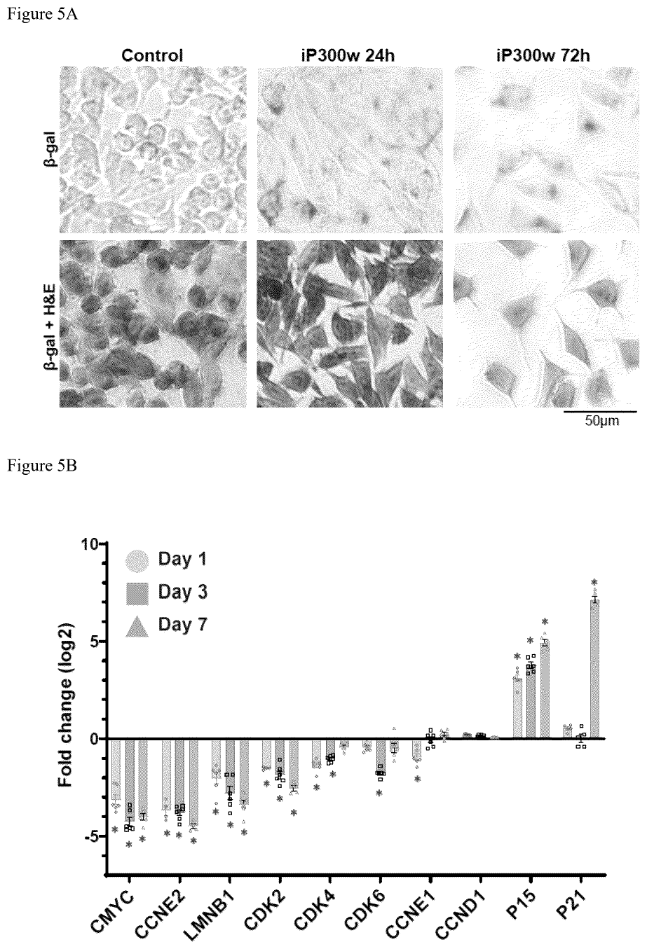

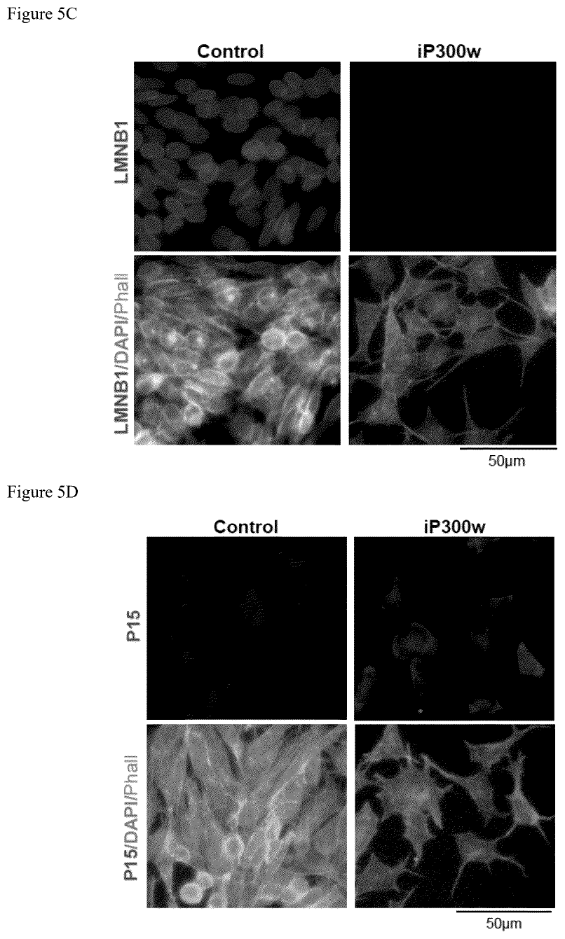

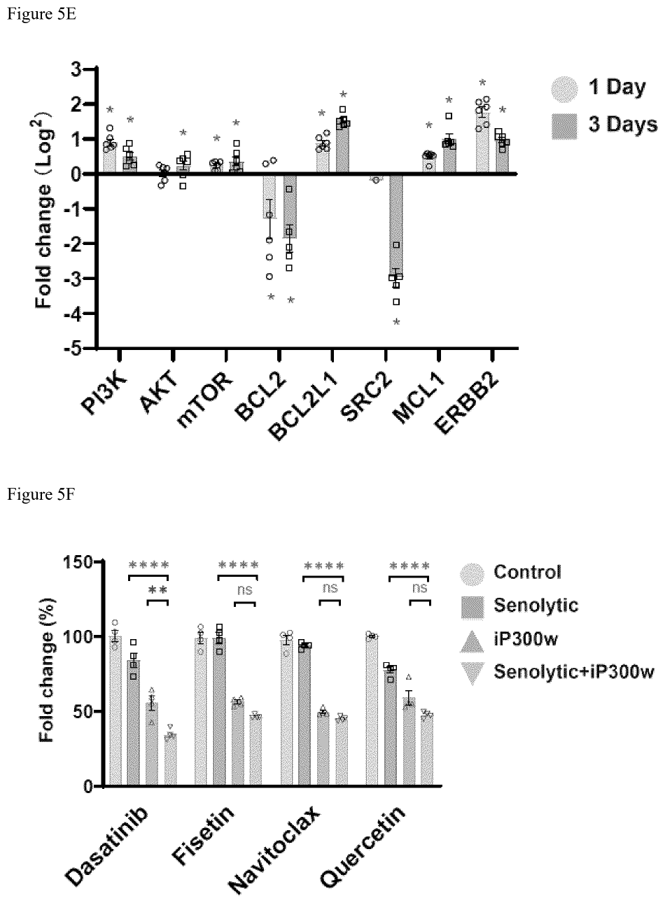

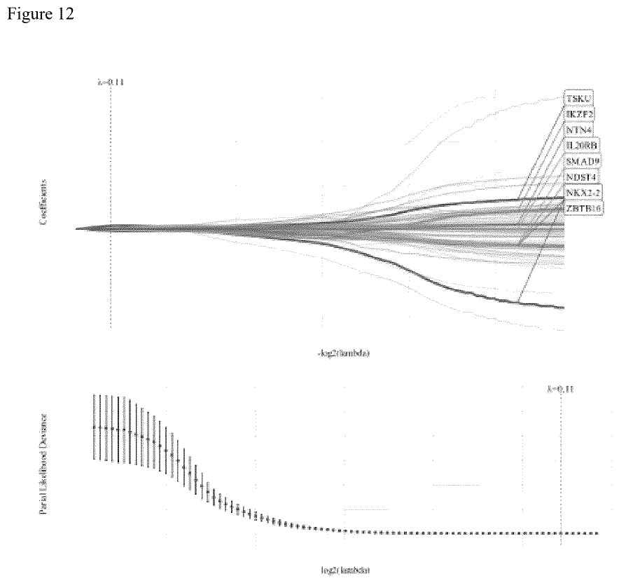

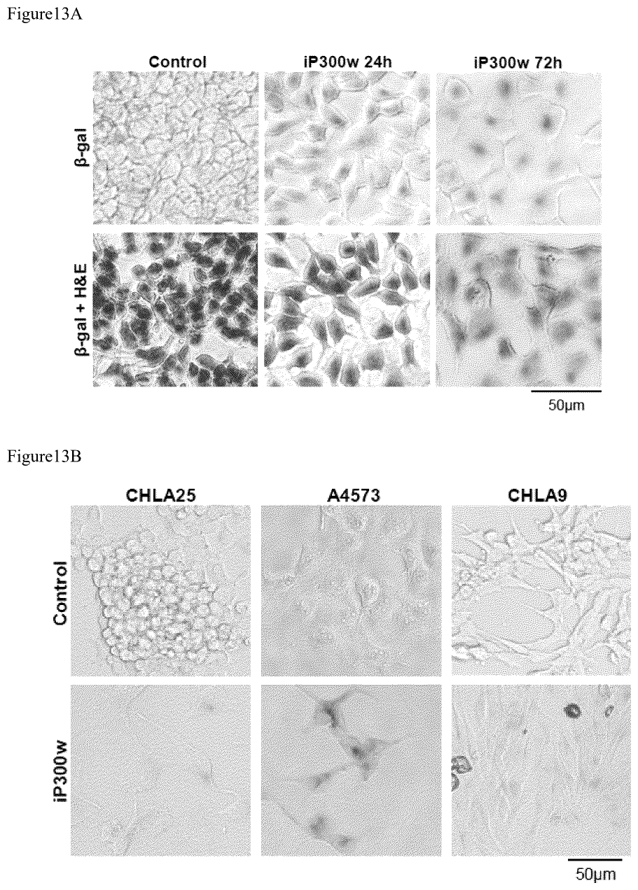

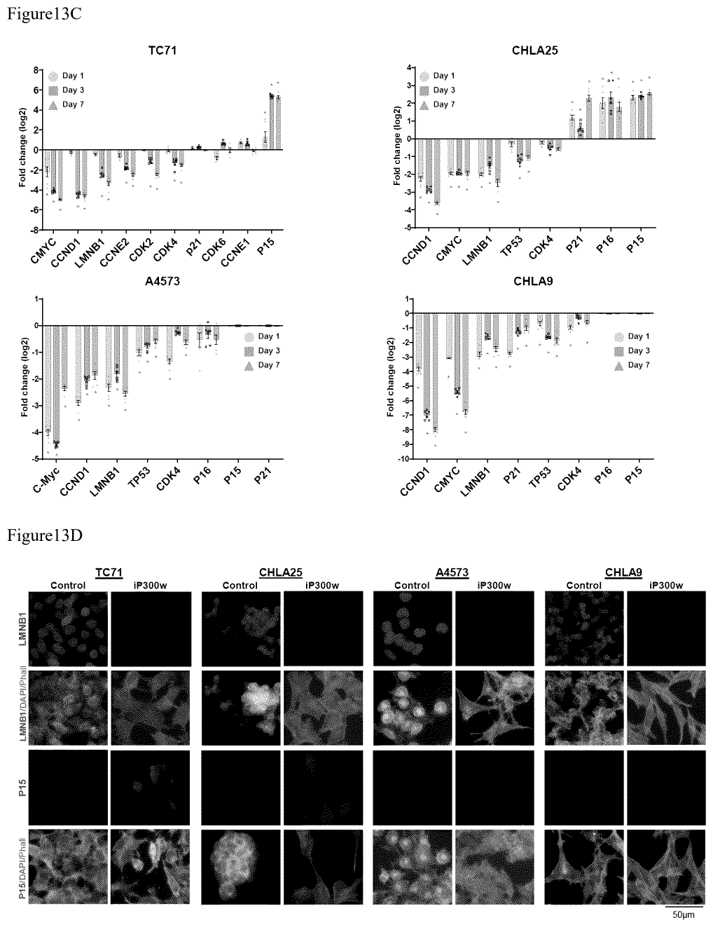

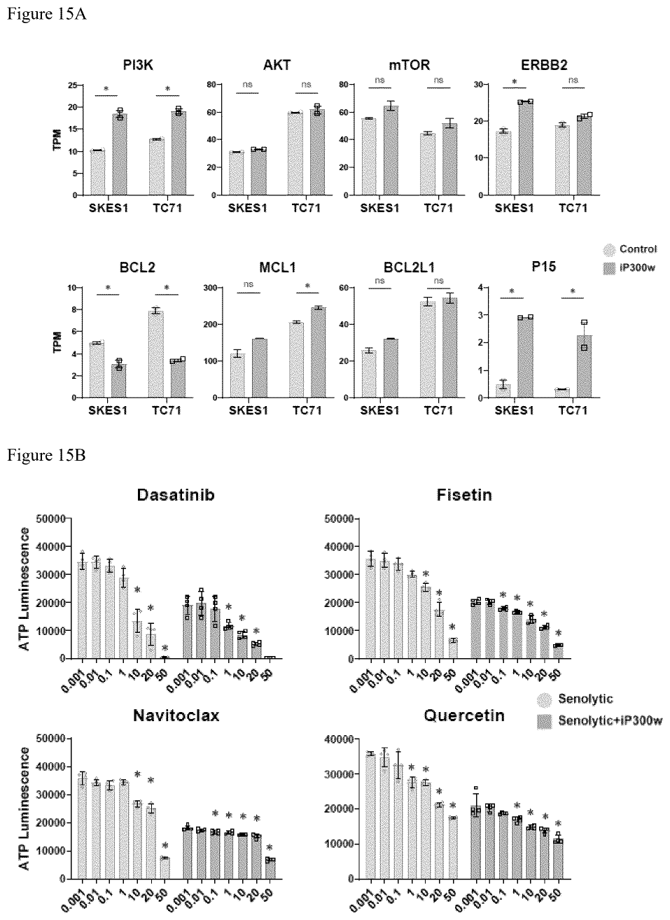

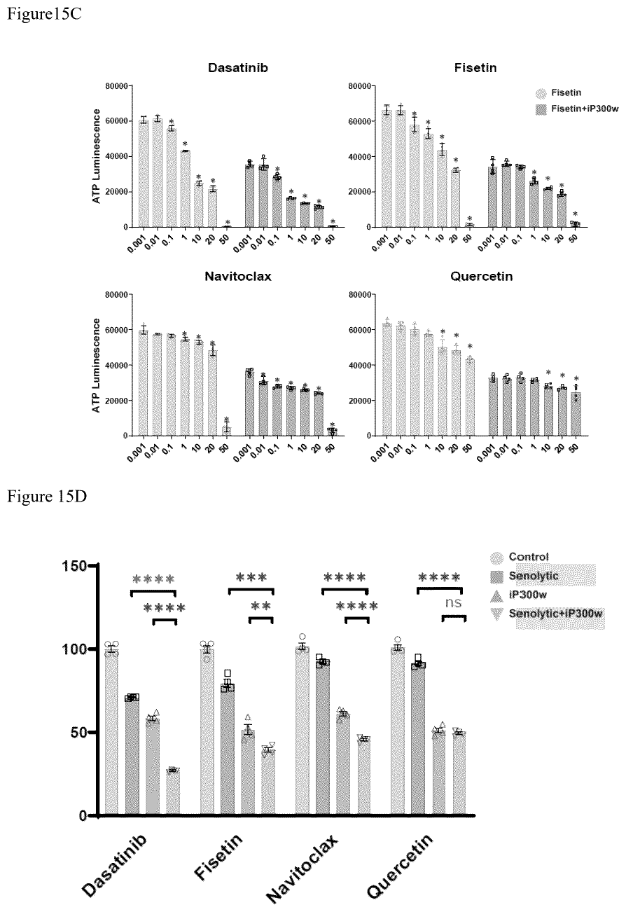

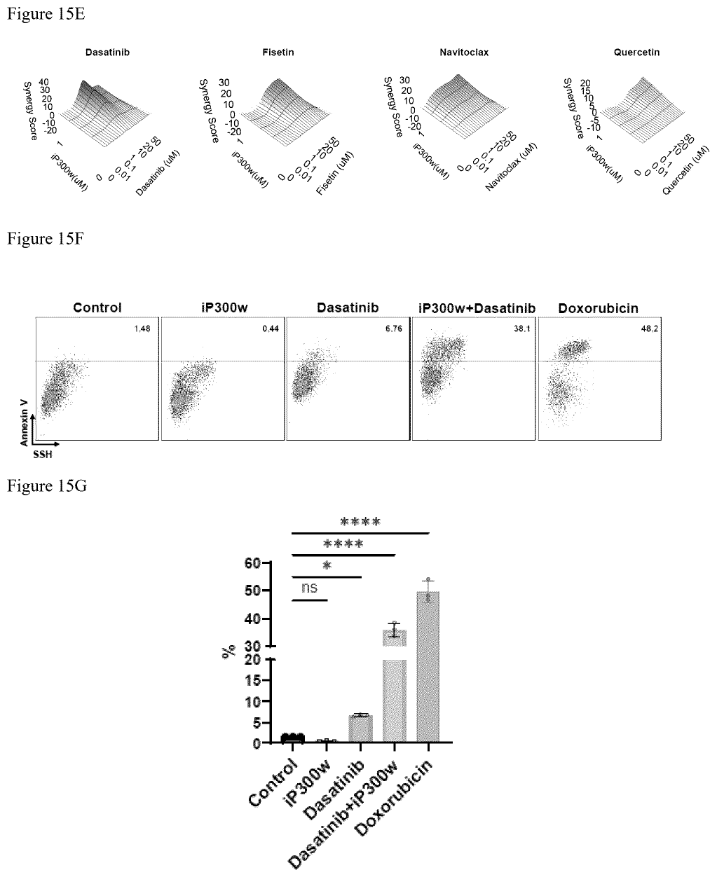

The findings, indicating iP300w treatment triggers rapid cell cycle arrest and morphological changes characteristic of senescent cells, coupled with KEGG pathway enrichment in Cellular Senescence, justified direct evaluation of the relationship between P300/CBP and senescence in ES. β-galactosidase (β-gal) staining, a widely recognized marker for cellular senescence, to assess senescence in ES cell lines was performed. A notable increase in β-Gal-positive cells was detected in SKES1 and TC71 24 hours after iP300w treatment, even in the absence of evident morphological changes at this stage (FIG. 5A; FIG. 13A). Both cell lines exhibited a pronounced increase in β-Gal-positive staining 72 hours after treatment. Concurrent Hematoxylin and Eosin (H&E) staining in SKES1 cells revealed significant morphological changes characteristic of senescence, such as enlarged and flattened cell structures, irregular shapes, and increased granularity (FIG. 5A; FIG. 13A). To further validate these findings, senescence induction by iP300w through 1-gal staining in additional EWS::FLI1-driven Ewing sarcoma cell lines, including CHLA25, A4573, and CHLA9 (FIG. 13B) was confirmed. The analysis to track early and late senescence-associated gene expression over a seven-day period in these treated cell lines was expanded. Although cell cycle gene alterations highlighted a significant impact on proliferation, it was surprising to observe that the early senescence markers P16 and P21 in most cases were not upregulated (FIG. 5B; FIG. 13C). Upon broadening the panel of senescence-related markers, a considerable decrease in LMNB1 expression in all ES cell lines or xenograft tumors treated with iP300w (FIG. 2I) was noted. This effect was further confirmed at the protein level by immunofluorescence (FIGS. 5C and D; FIG. 13D). Interestingly, only the ES cell lines harboring functional P53 mutations (SKES1, TC71, CHLA25) responded with P15 induction upon iP300w treatment was observed, whereas the cell lines with intact P53 (A4573 and CHLA9) did not show this response (FIG. 5B; FIG. 13C). Remarkably, alterations in the expression of P15 was observed as early as 4 hours into the treatment (FIG. 15A).

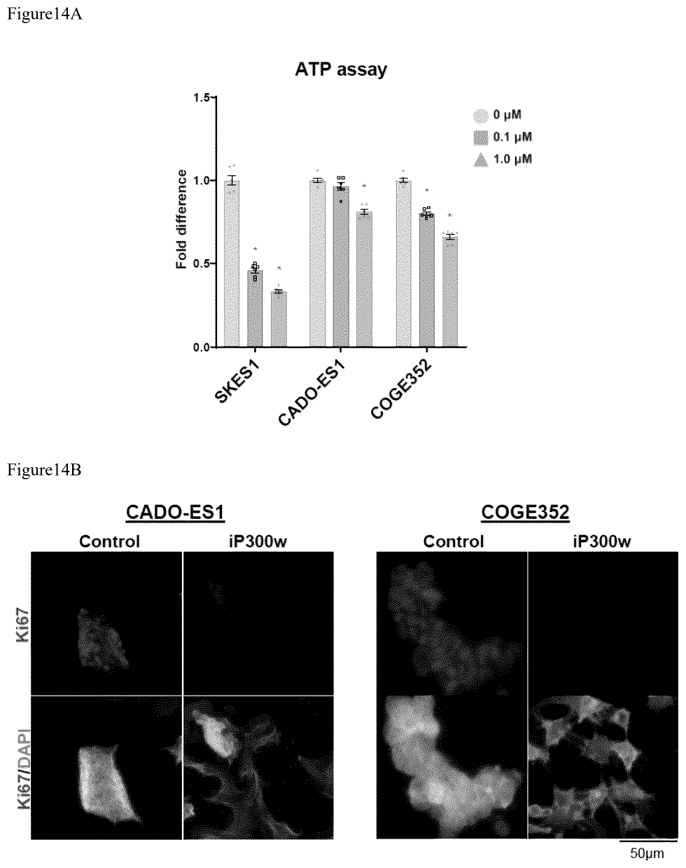

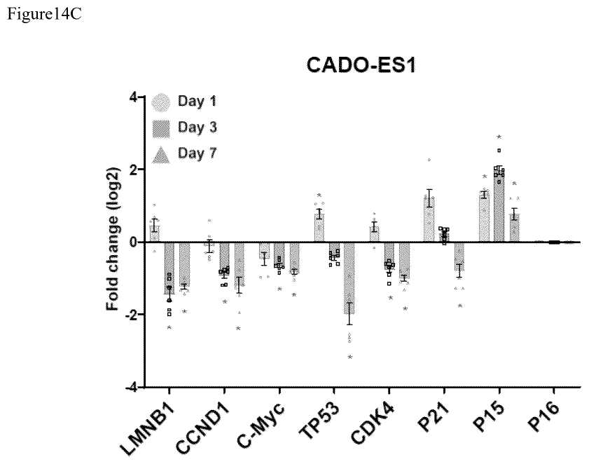

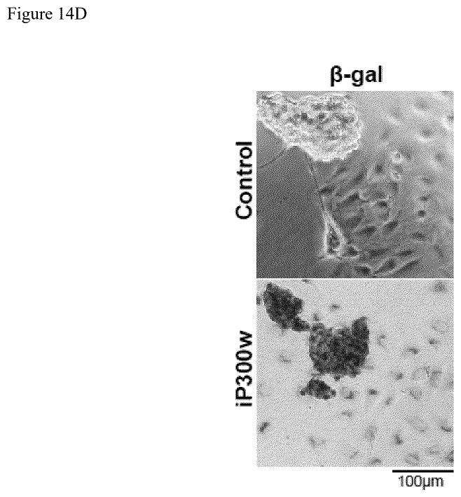

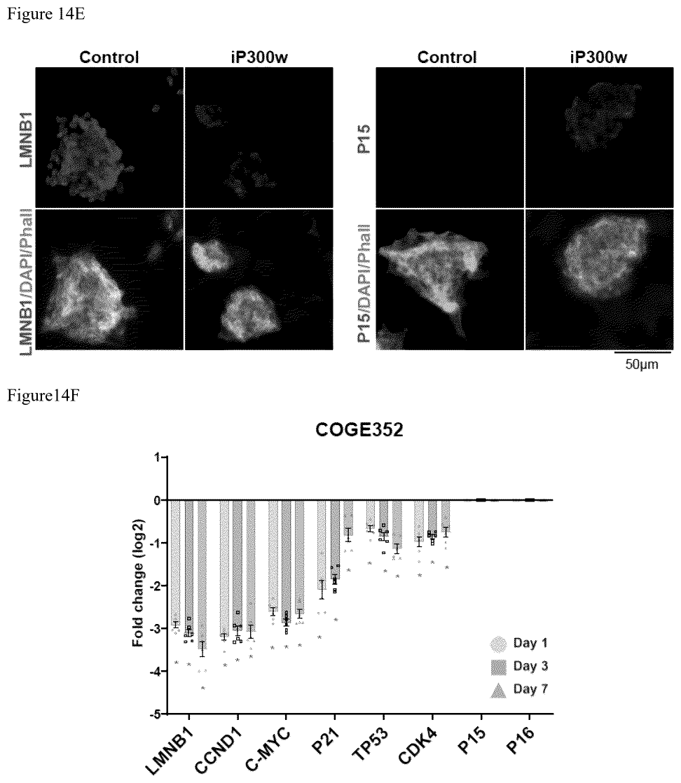

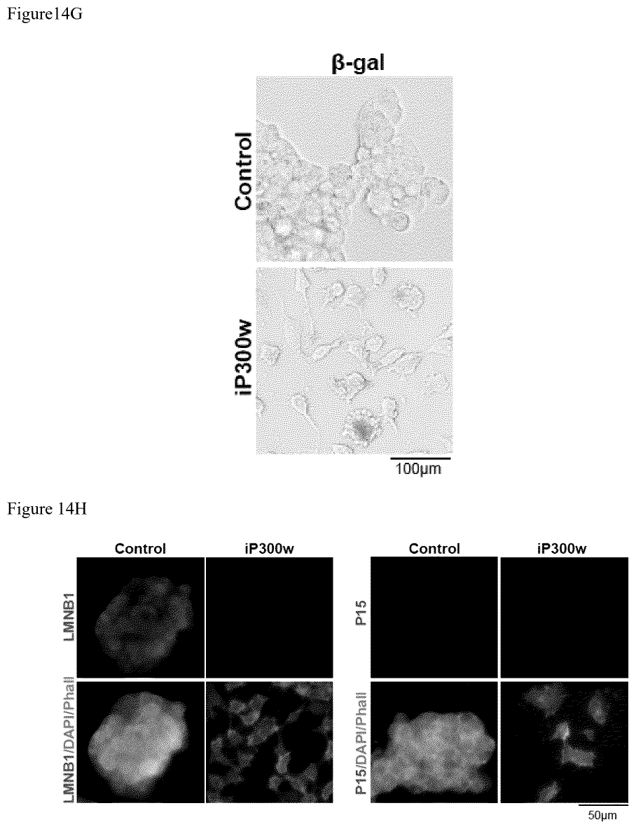

To determine whether P300/CBP inhibition also induces senescence in ES cell lines driven by EWS::ERG translocation, the analysis to the CADO-ES1 and COG-E-352 cell lines was extended. Initial results showed that iP300w treatment reduced cell viability in EWS::ERG cells, though this effect was less pronounced compared to EWS::FLI1 cell lines at 48 hours, as measured by the ATP assay (FIG. 13A). Notably, after extending the treatment to 72 hours, a complete loss of Ki67 staining, indicating a profound effect on cell proliferation was observed (FIG. 14B). Similarly to the EWS::FLI1 cell lines, both CADO-ES1 and COGE352 lines underwent senescence following iP300w treatment. This was evidenced by changes in cell morphology, expression of senescence-related genes, and β-galactosidase positivity (FIG. 13C-13H). Downregulation of LMNB1 was consistently observed as a hallmark of the senescence process (FIG. 13C, 13E, 13F, 13H).

It was then reasoned that if senescence is indeed one of the initial responses to P300/CBP inhibition, senolytics might facilitate the clearance of iP300w-treated ES cells. Senolytics comprise a group of drugs that target various senescence-related pathways [25]. To identify the most appropriate senolytic, the commonly affected PI3K and BCL-2 pathways by RT-qPCR were assessed. Gene expression analyses revealed a significant upregulation of PI3K, mTOR, MCL-1, and ERBB2, and a reduction of BCL-2 and SRC2 (FIG. 5E). RNA-seq data suggested signs of these changes were apparent as early as 4 hours following P300/CBP inhibition (FIG. 15A). Hence, for testing, Dasatinib and Fisetin, established senolytics targeting the PI3K pathways were chosen, while Navitoclax and Quercetin, known compounds that target BCL-2, served as negative controls [25-27]. To assess the potential synergistic effects of these senolytics when combined with iP300w, testing each senolytic across a concentration range from 0 to 5 μM was conducted. Dasatinib displayed an IC50 between 1-1 μM, while Fisetin and Navitoclax fell within the 20-5 μM range (FIG. 15B, 15C). Quercetin showed no effectiveness even at the highest tested concentrations. Combining iP300w with these senolytics significantly reduced cell viability, as evidenced by the percentage fold change compared to the control group in the ATP assay (FIG. 15B, 15C). Particularly noteworthy was the marked improvement observed with the combination of Dasatinib with iP300w that resulted in a 70% inhibition of cell viability at 1 μM concentrations for both agents (FIG. 5F; FIG. 15D). This underscores a potent synergistic effect when iP300w is used alongside selected senolytics. To quantitatively assess the synergistic effects of combining iP300w with senolytics, SynergyFinder Plus was utilized. Analysis revealed Dasatinib at 1 μM, when combined with 1 μM of iP300w, achieved the highest synergy score (FIG. 5G; FIG. 15E). After confirming that the combination of Dasatinib with iP300w exhibited the strongest synergistic effect, the ability of this combined treatment to induce apoptosis was evaluated. While Dasatinib alone caused only minimal apoptosis in SKES1 cells, the addition of iP300w significantly enhanced this effect, achieving levels comparable to those induced by Doxorubicin (FIG. 5H, 5I; FIG. 15F, 15G). Bringing these findings together, it was concluded that ES undergoes senescence as an early response to P300/CBP inhibition, characterized by alterations in LMNB1 and PI3K expression.

### Regulation of LMNB1 by the EWS::FLI1

The noncanonical senescence pathway induced by iP300w in ES prompted further investigation into the interplay between LMNB1/P15 and EWS::FLI1/P300/CBP transcriptional axis. To explore this, previously published high-resolution ChIP-Seq data obtained from studies examining the epigenetic effects of gain and loss of EWS::FLI1 function in ES and MCS was leveraged [8, 16].

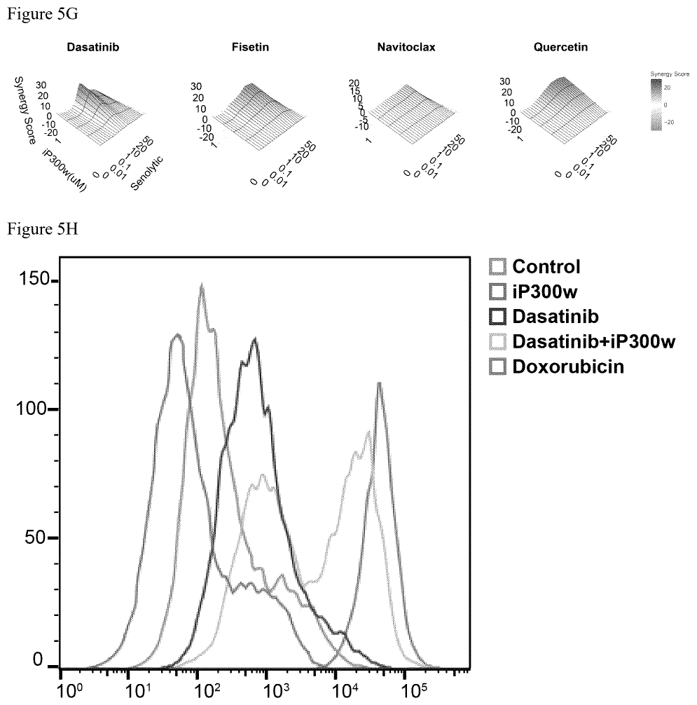

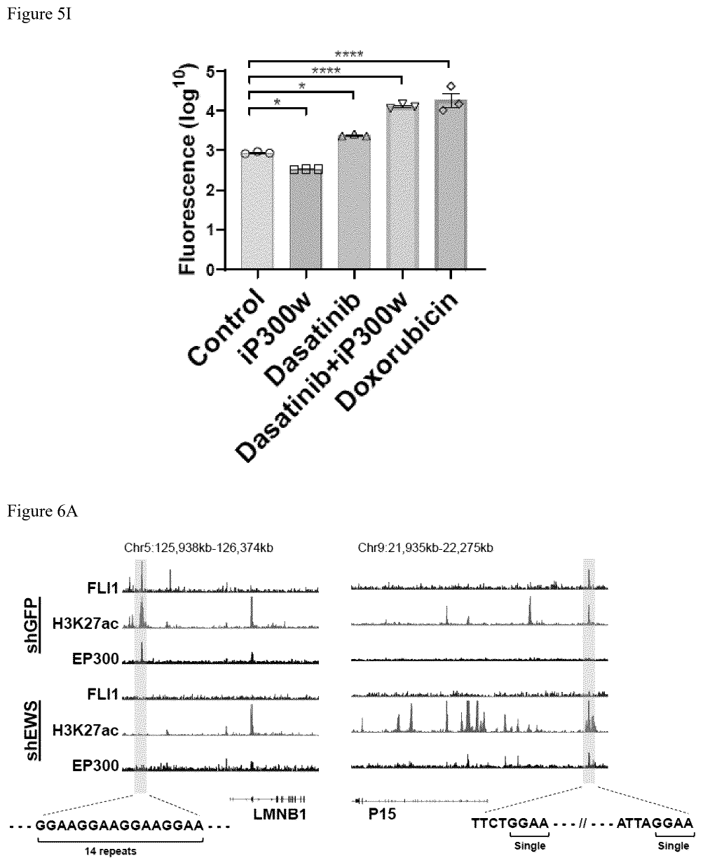

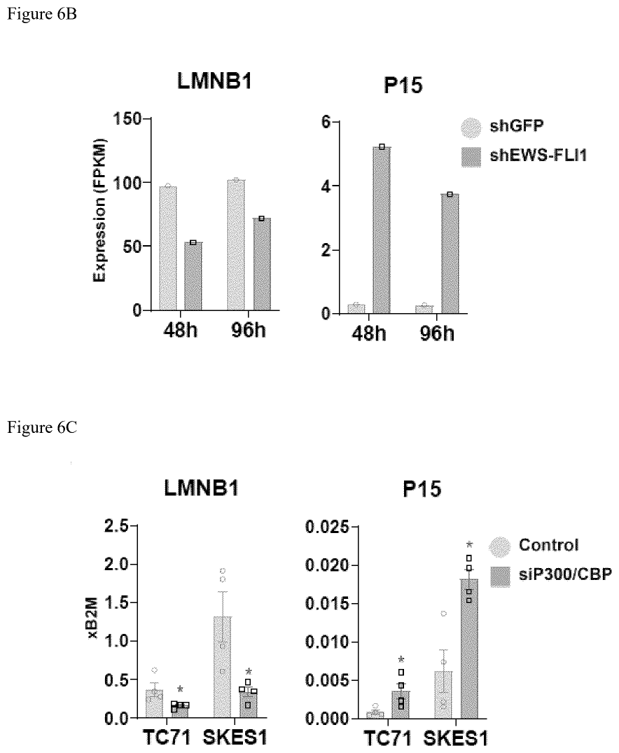

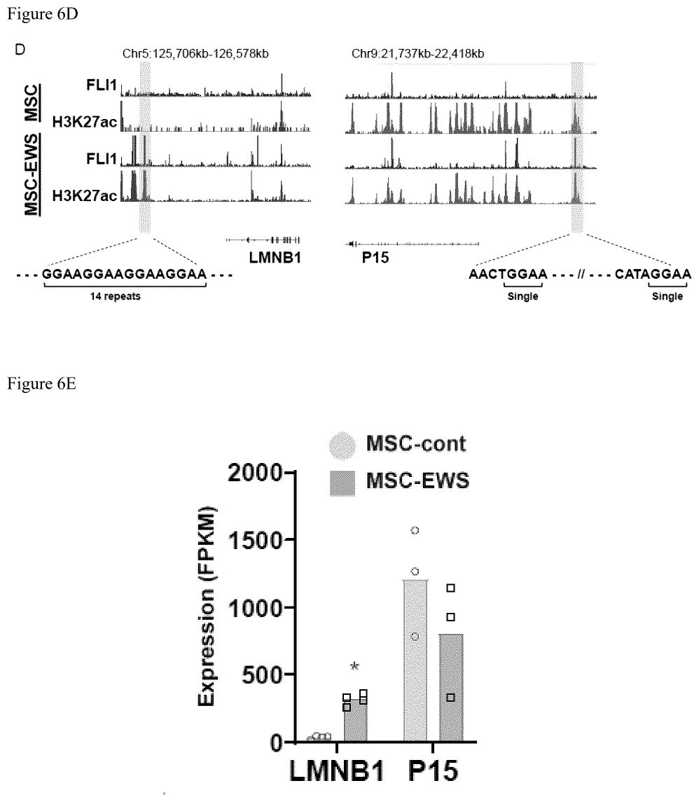

Analysis of ChIP-Seq data revealed EWS::FLI1 binding in the regulatory elements of both LMNB1 and P15. The binding region in LMNB1 comprises consecutive GGAA repeats (more than 14), which are associated with EWS::FLI1 transcriptional activation, while the binding site in P15 features a single GGAA motif that facilitates oncogene transcriptional repression (FIG. 6A). Notably, the EWS:FLI1 peak in LMNB1 is also associated with acetylated H3K27 and P300, indicating active chromatin. Upon EWS::FLI1 knockdown, the EWS:FLI1 binding is lost as expected, along with simultaneous loss of P300 binding and reduction of H3K27 acetylation (FIG. 6A). The epigenetic alterations in the LMNB1 enhancer are reflected in the transcriptional levels, as the gene is downregulated by EWS:FLI1 knockdown (FIG. 6B). Contrary to LMNB1, EWS::FLI1 binding of P15 was not accompanied by P300 and maintained low H3K27 acetylation. Upon EWS::FLI1 knockdown, the repression of the locus was lost, as indicated by increased acetylation and gene induction (FIGS. 6A and B). To confirm EWS::FLI1 transcriptionally regulates LMNB1 through P300/CBP, the data from experiments in which both proteins were knocked down was reassessed (FIG. 1). Indeed, the transcription of LMNB1 upon P300/CBP knockdown mirrored those observed with EWS::FLI1 knockdown (FIG. 6C).

Overexpression of EWS::FLI1 in non-permissive cells leads to induction of cell cycle arrest and cell death [14]. Therefore, it was hypothesized that the opposite mechanism might apply when EWS::FLI1 is expressed in a permissive environment. To investigate this, ChIP-seq data in which EWS::FLI1 was overexpressed in MSC to promote malignant transformation was retrieved [16, 28]. As predicted, EWS::FLI1 overexpression resulted in EWS::FLI1 binding to the repetitive GGAA motif, facilitating H3K27 acetylation and LMNB1 expression (FIGS. 6D and E). Additionally, EWS-FLI1 binds to the repressive single GGAA motif, leading to reduced H3K27 acetylation at the P15 locus (FIG. 6D). In summary, these findings demonstrate the EWS::FLI1/P300/CBP axis directly regulates the expression of key senescence-related genes LMNB1, promoting cell viability and preventing senescence in Ewing sarcoma and permissive cells.

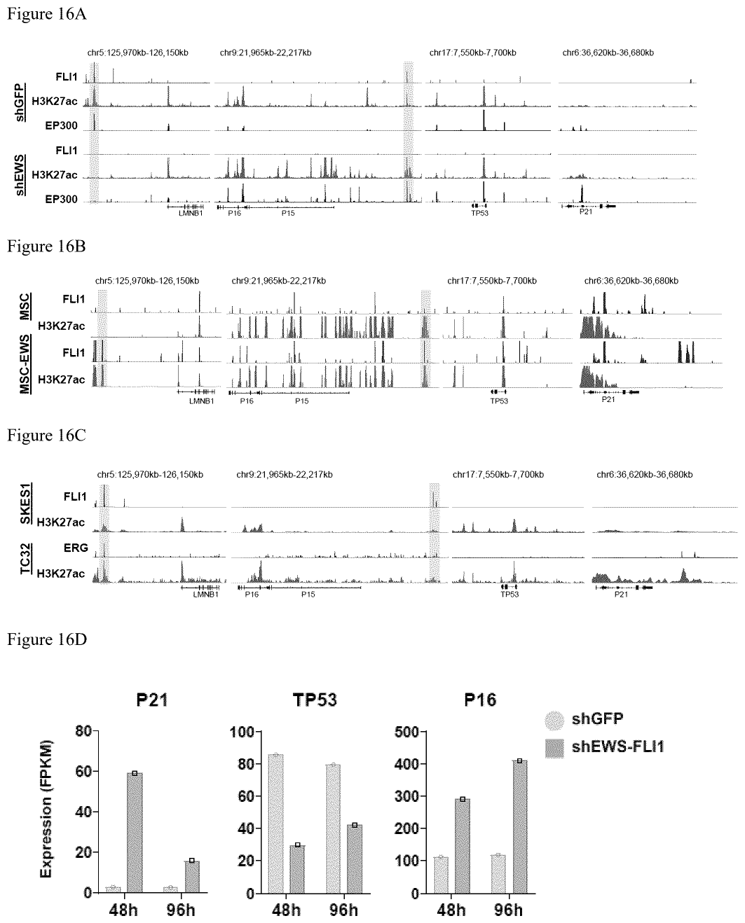

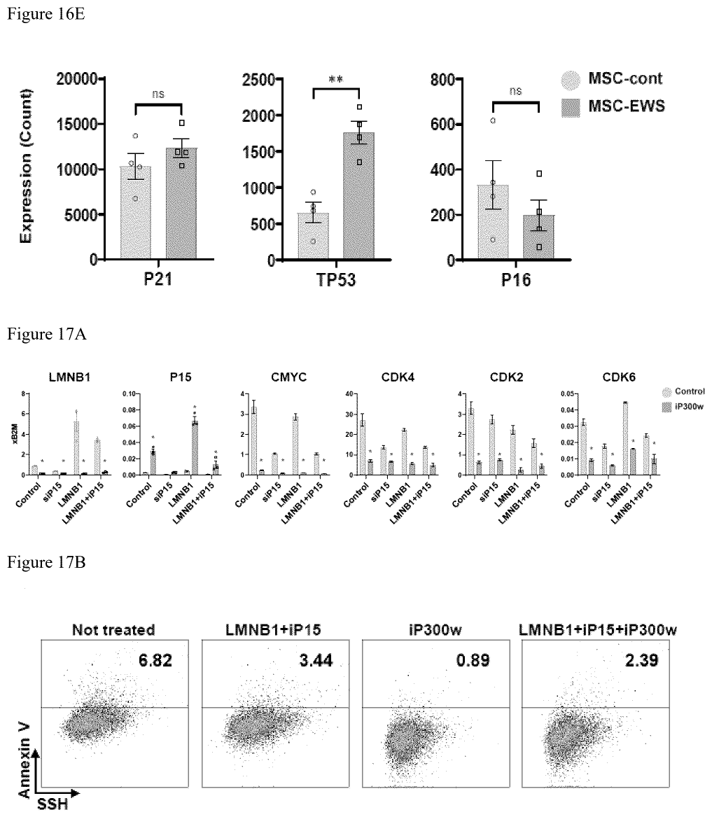

To further investigate whether EWS::FLI1 and EWS::ERG-mediated senescence evasion mechanisms operate independently of the canonical TP53 and P16/P21 pathways, the binding sites of these oncoproteins at their regulatory regions in SKNMC, SKES1, and TC32 cells was examined. Analysis revealed that both EWS::FLI1 and EWS::ERG consistently bind only to LMNB1, with no direct interactions observed with TP53, P16, or P21. This suggests that any transcriptional activity of P16, P21, and TP53 might be independent of these oncoproteins (FIG. 16A-16E). Additionally, the previously observed binding of EWS::FLI1 to the repressive motif of P15 in SKNMC was also identified in SKES1 cells, both of which have nonfunctional P53. Notably, EWS::FLI1 binding to P15 is not associated with P300 peaks, indicating a P300/CBP-independent regulatory mechanism (FIG. 16A). In summary, it has been demonstrated that both EWS::FLI1 and EWS::ERG cooperate with P300 to maintain LMNB1 expression and evade senescence-related processes in Ewing sarcoma.

LMNB1 and P15 Suppress Apoptosis and Sustain iP300w Induced Senescence

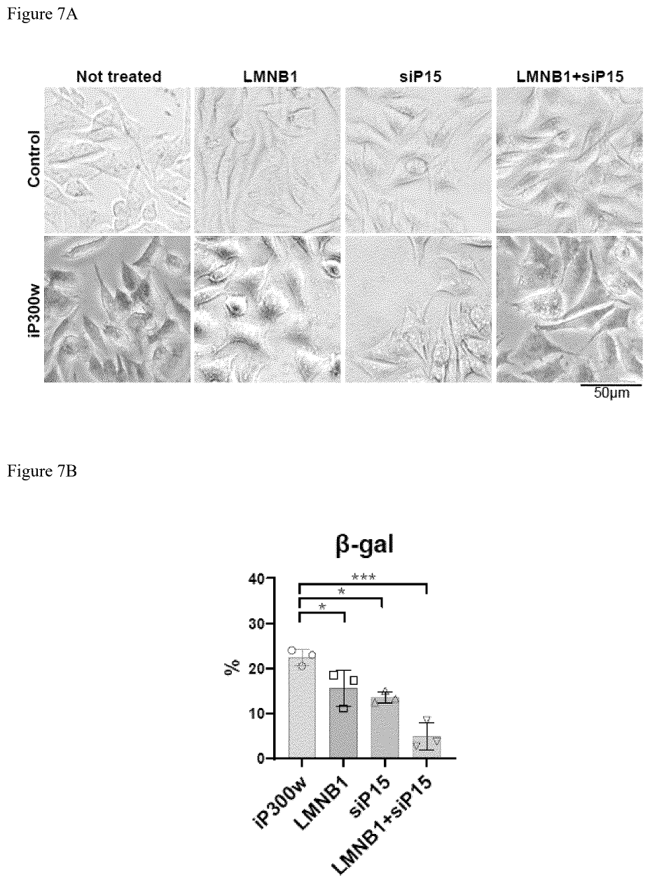

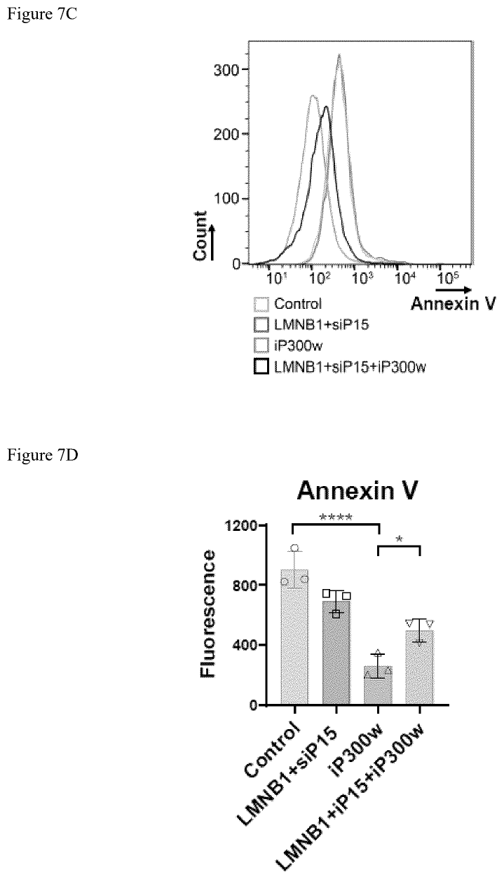

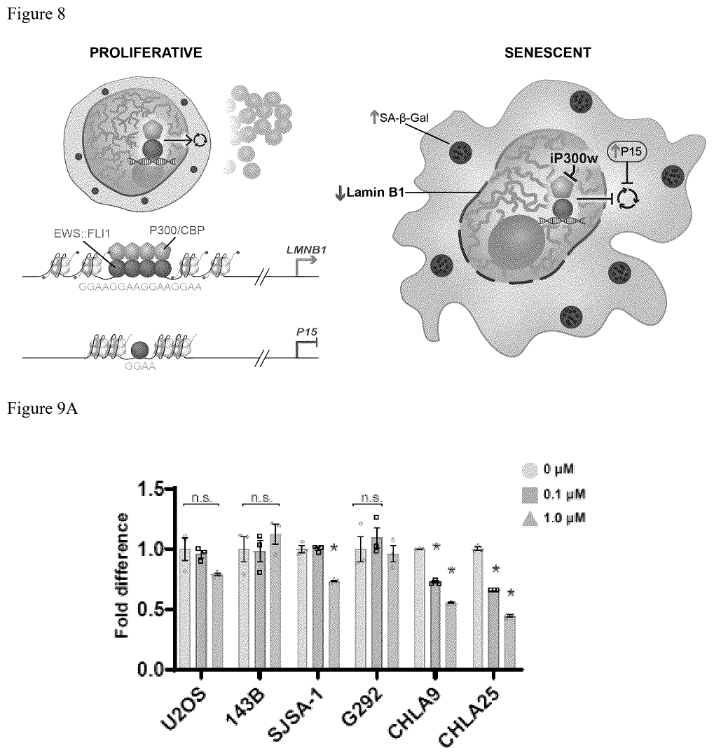

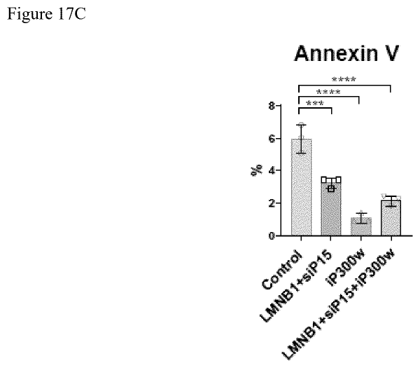

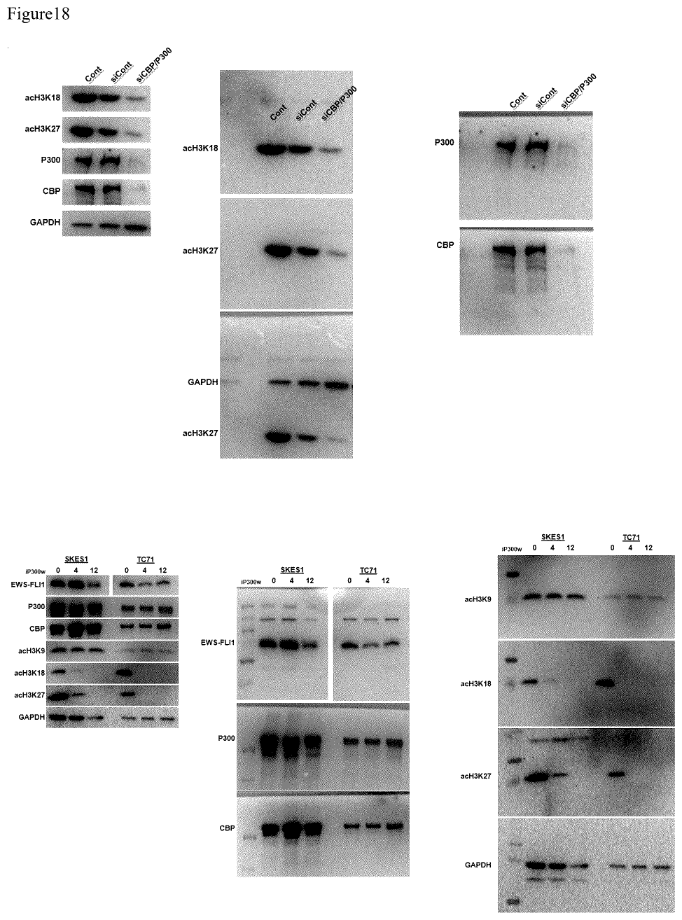

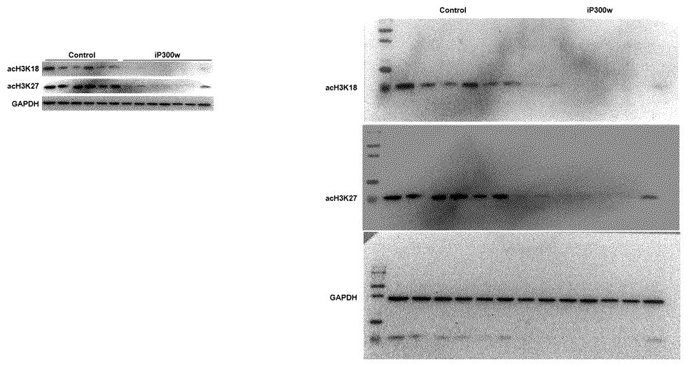

To assess the functional relevance of iP300w induced senescence mediated by LMNB1/P15 in ES cells with non-functional P53, gene rescue experiments were conducted. The assumption was that simultaneous overexpression of LMNB1 and knockdown of P15 would delay the senescence. To achieve constitutive LMNB1 overexpression, SKES1 cells were transduced with a lentiviral vector carrying LMNB1, while P15 was knocked down using siRNA (FIG. 17A). β-gal staining revealed a significant induction in 21% of the cells by 24 hours of iP300w treatment, a process notably reduced to 4% upon overexpression of LMNB1 and knockdown of P15 (FIGS. 7A and B).

Senescent cells evade apoptosis through a variety of mechanisms, such as enhancing anti-apoptotic pathways, modifying pro-survival signaling, and regulating apoptotic regulators, enabling them to persist in a senescent state [29]. Annexin V staining to evaluate the apoptotic status in LMNB1/P15 rescued cells was conducted. As anticipated, SKES1 cells treated with iP300w exhibited a significant reduction in fluorescence signal and a marked decrease in Annexin V-positive cells (FIG. 7C, D; FIG. 17B, 17C). However, following LMNB1 overexpression and P15 RNA interference, both the fluorescence intensity and the proportion of Annexin V-positive cells were partially reversed (FIG. 7C, D; FIG. 17B, 17C). These functional experiments provide direct evidence that LMNB1 and P15 play a critical role in iP300w-induced senescence.

### Discussion

This study unveils the critical mechanism of the EWS::FLI1/P300/CBP axis in maintaining ES viability and exposes its vulnerability for pharmacological targeting by the P300/CBP inhibitor, iP300w. Additionally, a novel mechanism by which EWS::FLI1 prevents ES or permissive MSC from undergoing senescence by controlling the expression of LMNB1 was elucidated. EWS::FLI1, together with P300/CBP induce LMNB1 transcription by binding to multiple GGAA repeats in its regulatory elements. P300/CBP inhibition rapidly inactivated EWS::FLI1 transcriptional activity, leading to the downregulation of LMNB1, consequently inducing cell senescence. Finally, that PI3K-targeting senolytics effectively eliminate iP300w-induced senescent ES cells, suggesting a novel, combined therapeutic approach was demonstrated.

While prior studies have shown EWS::FLI1 can recruit P300 to differentially regulate super enhancers [8], the role of P300/CBP in ES viability remains undefined [15]. To explore this, P300/CBP was initially knocked down using siRNA and demonstrated that the oncogenic activity of EWS::FLI1 largely depends on these acetyltransferases. Prompted by these initial results and recognizing the translation limitations of the siRNA approach [30], pharmacological targeting of P300/CBP, capitalizing on the new generation of more specific spirocyclic P300/CBP inhibitors, including iP300w was conducted [19, 31]. iP300w as an effective inactivator of DUX4, a pioneer transcriptional factor that recruits P300/CBP through its C-terminal domain and plays a key role in early embryonic development and facioscapulohumeral muscular dystrophy (FSHD) was described [31, 32]. The translocation of the DUX4 C-terminus transforms CIC from a transcriptional repressor to an activator, thereby driving the development of CIC::DUX4 sarcoma. Notably, iP300w displayed superior efficacy against CIC::DUX4 sarcoma compared to A-485 [18], another P300/CBP inhibitor from the same spirocyclic chemical series found to be effective against certain hematological malignancies, androgen receptor-positive prostate cancer, and NUT midline carcinoma [19, 33]. Likewise, iP300w demonstrated high efficacy against ES, evidenced by rapid induction of cell arrest and inactivation of EWS::FLI1 transcriptional activity, comparable to the effects seen with knockdown of EWS::FLI1 [23, 34]. The observed effect was specific to ES cells, with minimal reduction in cell viability noted in human myoblasts and osteosarcoma cell lines at the tested concentrations. Furthermore, the extent of global transcriptional alterations in ES cell lines treated with iP300w surpassed that observed in synovial sarcoma, rhabdomyosarcoma, or osteosarcomas. These findings underscore the pivotal role of P300/CBP in ES malignancy, as evidenced by a robust correlation between patient stratification based on specific EWS::FLI1/P300/CBP-related gene expression patterns and overall survival rates. Notably, this correlation demonstrates greater predictive power for survival outcomes than traditional clinical features [35]. Similarly, elevated levels of P300 have been identified as indicators of high malignancy and poor prognostic survival factors in liver, nasopharyngeal, and prostatic cancers [36-38].

Despite extensive research over the decades, treatment options for Ewing Sarcoma (ES) remain limited. FDA-approved drugs such as vincristine, doxorubicin, and cyclophosphamide show some effectiveness but lack specificity for the EWS::FLI1-driven malignancy[39-41]. These are drugs that target DNA replication and cell proliferation, leading to unintended effects on healthy cells. Ifosfamide, another commonly used chemotherapeutic agent for ES, is an alkylating agent that cross-links DNA strands [42]. It is associated with significant side effects, including renal toxicity, cardiotoxicity, and neurotoxicity. Etoposide and topotecan, both topoisomerase inhibitors, are used in the treatment of various cancers, including ES, but their use can also lead to serious side effects such as secondary leukemias and myelosuppression [43, 44]. Other drugs that have shown some effectiveness for ES, when used in combination with other treatments, include irinotecan, actinomycin D, regorafenib, clofarabine, cladribine, and trabectedin [40, 45-48]. Recently, efforts to target the EWSR1::FLI1 fusion or its downstream pathways have been largely unsuccessful. For instance, TK-216, a pharmacological agent designed to disrupt the interaction between EWSR1::FLI1 and RNA helicase A, demonstrated responses in 3 of 85 patients in a phase I/II clinical trial, with an overall disease control rate of 25% and a median duration of response of 25.5 months [49]. Similarly, trabectedin, a DNA minor groove-binding agent, has demonstrated minimal clinical activity as a single agent despite its effects on EWSR1::FLI1 transcriptional targets [50]. Consequently, there is a critical need for therapies capable of specifically targeting EWS::FLI1 activity. The current study demonstrates that inhibiting P300/CBP can inactivate the EWS::FLI1 oncogenic axis, making it a rationally targeted pharmacological approach for treating ES.

Pathway analyses of differentially expressed genes alerted by iP300w revealed activation of the senescence pathway early in the treatment. The senescence phenotype was further evidenced by altered cell morphology, reduced cell proliferation, β-gal positive staining, and changes in gene expression related to senescence. Senescence induced by P300/CBP inhibition, whether via small molecules or knockdown, has been previously observed in various cancers, such as melanoma, non-small cell lung cancer, and breast cancer [51-53]. This effect is primarily attributed to global changes of chromatin acetylation, with subsequent impact on the regulatory elements of senescence-related genes, among others [54]. Conversely, recent reports indicate P300 inhibition prevents age-related cellular senescence, primarily driven by P300, inducing a dynamic, hyper-acetylated chromatin state and promoting the formation of active enhancer elements in the non-coding genome [55]. The rapid induction of senescence in ES by iP300w suggests a more specific interplay. This prompted further mechanistic exploration, uncovering a novel mechanism by which EWS::FLI1 directly and concurrently regulates the expression of LMNB1. Surprisingly, the classical senescence pathways typically mediated by P16 or P53/P21 were not affected by iP300w in ES [56, 57].

EWS::FLI1 exhibits distinct binding preferences depending on the arrangement of GGAA motifs within regulatory regions. When encountering repetitive GGAA motifs, EWS:FLI1 tends to promote gene activation. In contrast, when encountering single GGAA motifs, it tends to facilitate gene repression [8, 16]. A novel mechanism by which EWS::FLI1 directly abrogates senescence by activating LMNB1 through binding to repetitive GGAA motifs in its regulatory elements, thereby recruiting P300 and inducing H3K27 acetylation was found. Conversely, in the subset of cell lines harboring P53 mutation, it inhibits P15 independently of P300 through sites containing a single GGAA motif Previous studies have suggested various mechanisms by which EWS::FLI1 indirectly evades senescence, such as destabilizing P27 via Skp2 and suppressing P53 and P21 through the NOTCH signaling pathway [58-60]. It is well-documented that overexpression of EWS::FLI1 induces growth arrest and apoptosis in the majority of cell types, except for MSCs and neuroectodermal cells [12]. However, deletion of p53, p19, and p16 in mouse embryonic fibroblasts attenuates apoptosis and promotes EWS::FLI1-driven transformation [14, 61]. Intriguingly, that EWS::FLI1 directly controls LMNB1 and P15 in permissive MSC, potentially averting cell arrest, preventing senescence and fostering malignant transformation was also discovered. Loss of LMNB1 expression has been a well-documented trigger of senescence, a cellular state often characterized by the accumulation of P15 protein [62, 63]. Additionally, LMNB1 affects both proliferation and senescence, potentially through a ROS signaling pathway, leading to subsequent changes in chromatin, such as the enrichment of H3K4me3 and H3K27me3 [55, 64]. The functional relevance of LMNB1 and P15 in iP300w-mediated ES senescence through gene rescue experiments was further confirmed. By overexpressing LMNB1 and knocking down P15 in treated cells, the reconstitution of LMNB1 and P15 expression delayed iP300w-induced senescence and promoted apoptosis was observed. This was evidenced by a reduction in β-gal staining and an increase in Annexin V positive cells. Resistance of senescent cells to apoptosis occurs primarily through alterations in P53 [65]. They maintain low levels of P53 due to decreased stabilization and exhibit senescence-specific post-translational modifications of P53. These adaptations prevent the activation of P53-mediated apoptotic pathways, even in the presence of typical apoptotic stimuli like actinomycin D, low-dose cisplatin, and staurosporine [65].

Building on the success of inducing senescence in breast and lung cancer xenografts, followed by elimination with senolytics [66], a comparable strategy to target iP300w-treated ES cells was explored. The study revealed that P300/CBP inhibition activates the PI3K and suppresses BCL-2 pathways. To exploit this vulnerability, Dasatinib, a senolytic targeting the PI3K pathway was selected, for the targeted elimination of senescent cells [25]. Conversely, senolytics targeting the BCL-2 pathway, like Navitoclax [25], were completely ineffective in eliminating the senescent cells, further solidifying the involvement of the PI3K pathway. Dasatinib, a tyrosine kinase inhibitor, functions by inhibiting SRC or RTKs, consequently affecting the PI3K/AKT pathway [67]. The activation of the PI3K/AKT pathway broadens the target scope of Dasatinib, resulting in a synergistic effect when combined with iP300w. However, the initial decrease in BCL-2 expression in senescent cells might suggest a reduced target scope for BCL-2-targeting senolytics like Fisetin, Navitoclax, and Quercetin [25]. Nonetheless, the subsequent increase in MCL-1 levels can provide a compensatory survival mechanism for these cells. MCL-1, unique among the BCL-2 family, is responsible for conferring resistance to chemotherapeutic agents [26]. The upregulation of MCL-1 could attenuate the effects of BCL-2 inhibition, potentially accounting for the reduced synergy observed with P300/CBP inhibitors [26].

In conclusion, a novel mechanism by which EWS::FLI1 evades cellular senescence, pinpointing EWS:FLI1/P300/CBP as a vulnerable target in ES has been identified. Furthermore, the feasibility of pharmacological targeting of P300/CBP in ES has been demonstrated. While the pharmacological compound iP300w was utilized in this study, similar effects could potentially be achieved with other recently discovered P300/CBP inhibitors such as A-485, CCS1477, NE02734, and B026 which have shown effectiveness against various malignancies, with some even undergoing clinical trials [19, 68-70]. In addition, a more potent approach that integrates P300/CBP inhibition with senolytic agents to target newly senescent cells for elimination is proposed. By employing this dual approach, successful methodologies used in other cancer contexts is echoed and position it as a forward-looking avenue for ES treatment. This study delineates the critical roles of LMNB1 within this axis and demonstrates the efficacy of combining P300/CBP inhibition with senolytics, illuminating a potentially transformative approach to ES therapy. It underscores the intricate interplay of molecular pathways in ES and highlights the promising therapeutic implications of their modulation.

### Example 2

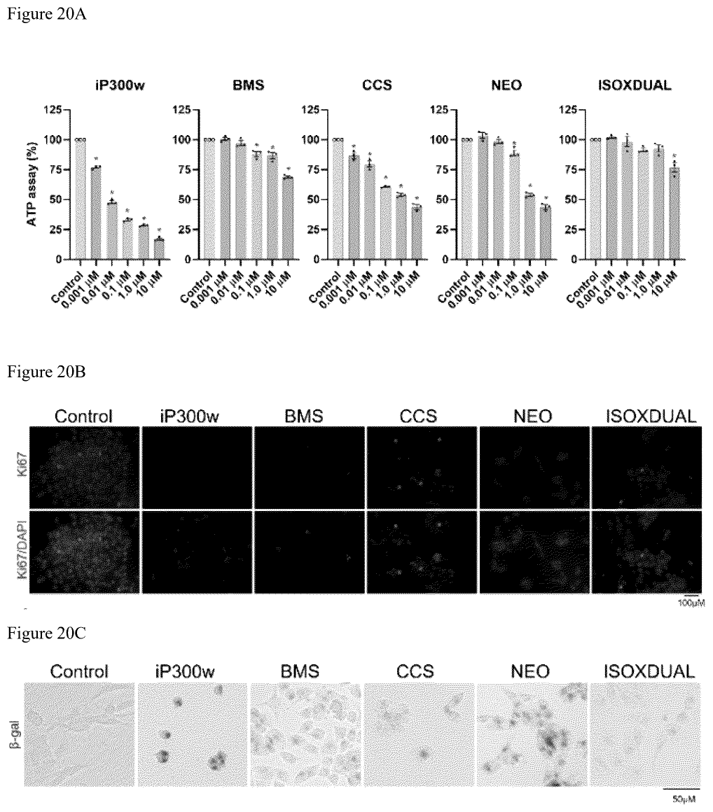

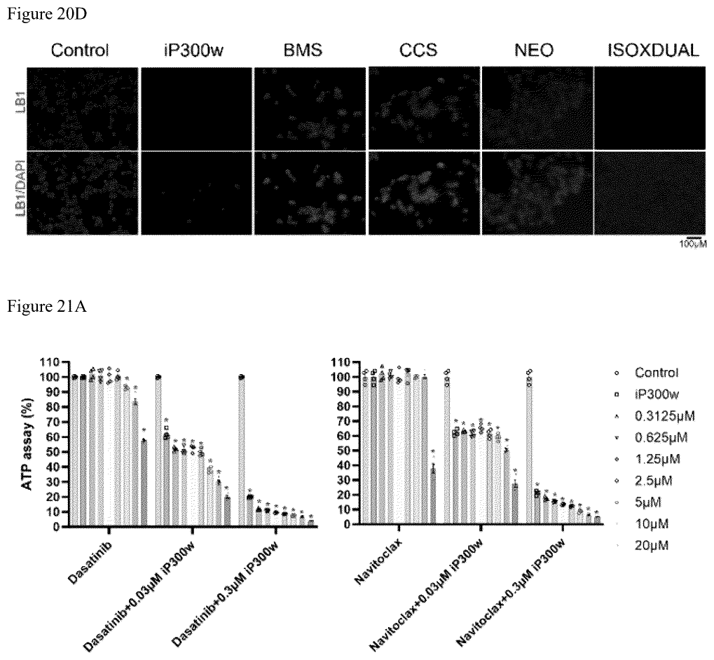

To assess whether P300/CBP inhibition constrains CIC::DUX4 sarcoma growth and creates a window for selective clearance, X1 cells were exposed for 72 hours to iP300w, BMS-986158, CCS1477, NE02734, and ISOX-DUAL across 0.001-10 μM. ATP-based viability measurements showed a concentration-dependent decrease with all agents (FIG. 20A). iP300w produced statistically significant reduction at the lowest concentration tested, whereas the other inhibitors required higher concentrations to reach significance. One-way ANOVA was applied for each compound across concentrations, with post hoc comparisons to the matched vehicle control. For follow-on assays, doses were set from prior sensitivity and approximate IC₅₀ information to achieve biological activity without nonspecific toxicity, using 0.1 μM iP300w and 1. μM for the other compounds. Consistent with the ATP data, Ki67 staining after 72 hours indicated near-complete loss of proliferative nuclei with iP300w and partial reduction with the other agents (FIG. 20B). Senescence-associated β-galactosidase positivity (FIG. 20C) and decreased LMNB1 staining (FIG. 20D) were observed under inhibitor treatment, indicating that reduced viability coincided with senescence-associated growth arrest rather than acute cytotoxicity.

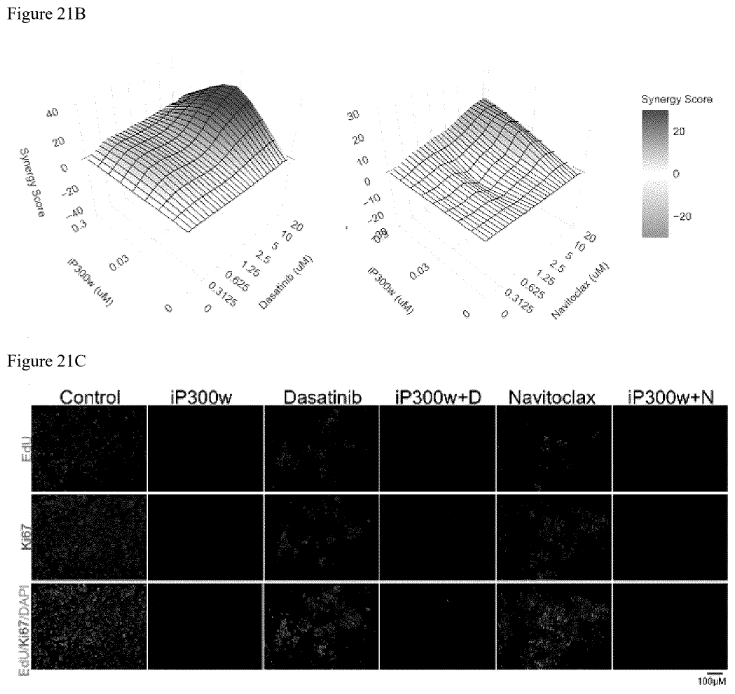

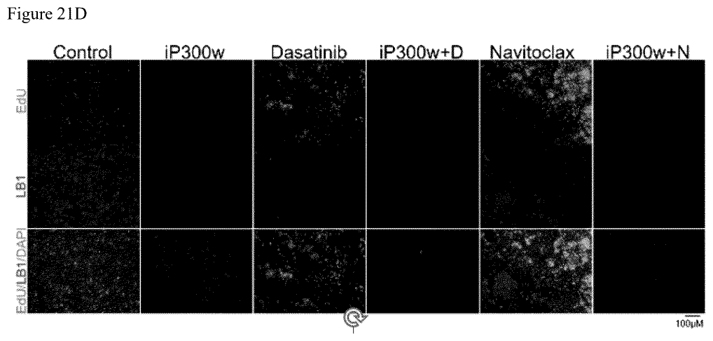

Whether senolytics agents could exploit this induced state were then accessed. For combination viability mapping, X1 cells first received 72 hours of iP300w at 0, 0.03, or 0.3 μM, after which cultures were maintained for an additional 72 hours with the same iP300w concentration in co-treatment with graded concentrations of Dasatinib or Navitoclax at 0, 0.3125, 0.625, 1.25, 2.5, 5, 10, and 2 μM (FIG. 21A). Viability was measured at the end of the second 72-hour interval. Two-way ANOVA was applied to test for effects of iP300w, senolytics concentration, and their interaction, with internal comparisons made to the controls Single-agent senolytics displayed concentration-dependent effects and reached statistical significance at higher doses. Across matched dose pairs, the combinations with ongoing iP300w produced greater decreases in viability than either agent alone. Interaction analysis with SynergyFinder Plus using the Highest Single Agent reference yielded positive synergy scores for iP300w with both Dasatinib and Navitoclax (FIG. 21B).

To evaluate therapeutic efficacy beyond exposure and to test whether combination treatment prevents proliferative recovery after drug removal, the sequence to include a defined recovery phase was extended. Cells received 72 hours of iP300w to induce the senescent-like state, followed by 72 hours of co-treatment with iP300w and senolytics agents, and were then transferred to drug-free medium for 72 hours. This design distinguishes transient growth arrest during treatment from depletion of the proliferative reservoir that would otherwise re-enter the cell cycle after withdrawal. During recovery, EdU was introduced once at 24 hours and removed after 8 hours to provide a pulse label of DNA synthesis (FIG. 21C). At the endpoint, the EdU incorporation, Ki67 expression, LMNB1 levels, and nuclear counts by DAPI were assessed. Following the iP300w plus senolytics sequence, EdU and Ki67 signals were not detected, LMNB1 remained decreased, and DAPI-based nuclear counts were lower than in the corresponding sequences without senolytics agents, indicating prevention of proliferative recovery following withdrawal (FIG. 21D).

### DOCUMENTS

1. Hesla, A. C., A. Papakonstantinou, and P. Tsagkozis, Current Status of Management and Outcome for Patients with Ewing Sarcoma. Cancers (Basel), 2021. 13(6).
2. Jurgens, H. and U. Dirksen, Ewing sarcoma treatment. Eur J Cancer, 2011. 47 Suppl 3: p. S366-7.
3. Esiashvili, N., M. Goodman, and R. B. Marcus, Jr., Changes in incidence and survival of Ewing sarcoma patients over the past 3 decades: Surveillance Epidemiology and End Results data. J Pediatr Hematol Oncol, 2008. 30(6): p. 425-30.
4. Subbiah, V., et al., Ewing's sarcoma: standard and experimental treatment options. Curr Treat Options Oncol, 2009. 10(1-2): p. 126-40.
5. Ghisoli, M., et al., Three-year Follow up of GMCSF/bi-shRNA(furin) DNA-transfected Autologous Tumor Immunotherapy (Vigil) in Metastatic Advanced Ewing's Sarcoma. Mol Ther, 2016. 24(8): p. 1478-83.
6. Riggi, N., M. L. Suva, and I. Stamenkovic, Ewing's Sarcoma. N Engl J Med, 2021. 384(2): p. 154-164.
7. Lin, L., et al., Super-enhancer-associated MEIS1 promotes transcriptional dysregulation in Ewing sarcoma in co-operation with EWS-FLI1. Nucleic Acids Res, 2019. 47(3): p. 1255-1267.
8. Riggi, N., et al., EWS-FLI1 utilizes divergent chromatin remodeling mechanisms to directly activate or repress enhancer elements in Ewing sarcoma. Cancer Cell, 2014. 26(5): p. 668-681.
9. Shulman, D. S., et al., An international working group consensus report for the prioritization of molecular biomarkers for Ewing sarcoma. NPJ Precis Oncol, 2022. 6(1): p. 65.
10. Zollner, S. K., et al., Ewing Sarcoma-Diagnosis, Treatment, Clinical Challenges and Future Perspectives. J Clin Med, 2021. 10(8).
11. Balamuth, N.J. and R. B. Womer, Ewing's sarcoma. Lancet Oncol, 2010. 11(2): p. 184-92.
12. Riggi, N., et al., EWS-FLI-1 expression triggers a Ewing's sarcoma initiation program in primary human mesenchymal stem cells. Cancer Res, 2008. 68(7): p. 2176-85.
13. Evdokimova, V., et al., Current State of Immunotherapy and Mechanisms of Immune Evasion in Ewing Sarcoma and Osteosarcoma. Cancers (Basel), 2022. 15(1).
14. Lessnick, S. L., C. S. Dacwag, and T. R. Golub, The Ewing's sarcoma oncoprotein EWS/FLI induces a p53-dependent growth arrest in primary human fibroblasts. Cancer Cell, 2002. 1(4): p. 393-401.
15. Chen, Q., et al., Histone acetyltransferases CBP/p300 in tumorigenesis and CBP/p300 inhibitors as promising novel anticancer agents. Theranostics, 2022. 12(11): p. 4935-4948.
16. Boulay, G., et al., Cancer-Specific Retargeting of BAF Complexes by a Prion-like Domain. Cell, 2017. 171(1): p. 163-178 e19.
17. Garcia-Dominguez, D. J., et al., Selective inhibition of HDAC6 regulates expression of the oncogenic driver EWSR1-FLI1 through the EWSR1 promoter in Ewing sarcoma. Oncogene, 2021. 40(39): p. 5843-5853.
18. Bosnakovski, D., et al., Inactivation of the CIC-DUX4 oncogene through P300/CBP inhibition, a therapeutic approach for CIC-DUX4 sarcoma. Oncogenesis, 2021. 10(10): p. 68.
19. Lasko, L. M., et al., Discovery of a selective catalytic p300/CBP inhibitor that targets lineage-specific tumours. Nature, 2017. 550(7674): p. 128-132.
20. Kawai, H., et al., Dual role of p300 in the regulation of p53 stability. J Biol Chem, 2001. 276(49): p. 45928-32.
21. Shimizu, K., et al., Interplay between protein acetylation and ubiquitination controls MCL1 protein stability. Cell Rep, 2021. 37(6): p. 109988.
22. Waddell, A., et al., p300 KAT Regulates SOX10 Stability and Function in Human Melanoma. Cancer Res Commun, 2024. 4(8): p. 1894-1907.
23. Orth, M. F., et al., Systematic multi-omics cell line profiling uncovers principles of Ewing sarcoma fusion oncogene-mediated gene regulation. Cell Rep, 2022. 41(10): p. 111761.
24. Zhang, J., et al., The International Cancer Genome Consortium Data Portal. Nat Biotechnol, 2019. 37(4): p. 367-369.
25. Chaib, S., T. Tchkonia, and J. L. Kirkland, Cellular senescence and senolytics: the path to the clinic. Nat Med, 2022. 28(8): p. 1556-1568.
26. Lee, A., et al., Synergism of a novel MCL-1 downregulator, acriflavine, with navitoclax (ABT-263) in triple-negative breast cancer, lung adenocarcinoma and glioblastoma multiforme. Int J Oncol, 2022. 60(1).
27. Mayer, E. L., et al., A phase 2 trial of dasatinib in patients with advanced HER2-positive and/or hormone receptor-positive breast cancer. Clin Cancer Res, 2011. 17(21): p. 6897-904.
28. Riggi, N., et al., Development of Ewing's sarcoma from primary bone marrow-derived mesenchymal progenitor cells. Cancer Res, 2005. 65(24): p. 11459-68.
29. Hu, L., et al., Why Senescent Cells Are Resistant to Apoptosis: An Insight for Senolytic Development. Front Cell Dev Biol, 2022. 10: p. 822816.
30. Ali Zaidi, S. S., et al., Engineering siRNA therapeutics: challenges and strategies. J Nanobiotechnology, 2023. 21(1): p. 381.
31. Bosnakovski, D., et al., A novel P300 inhibitor reverses DUX4-mediated global histone H3 hyperacetylation, target gene expression, and cell death. Sci Adv, 2019. 5(9): p. eaaw7781.
32. Choi, S. H., et al., DUX4 recruits p300/CBP through its C-terminus and induces global H3K27 acetylation changes. Nucleic Acids Res, 2016. 44(11): p. 5161-73.
33. Zhang, X., et al., Therapeutic targeting of p300/CBP HAT domain for the treatment of NUT midline carcinoma. Oncogene, 2020. 39(24): p. 4770-4779.
34. Chansky, H. A., et al., Targeting of EWS/FLI-1 by RNA interference attenuates the tumor phenotype of Ewing's sarcoma cells in vitro. J Orthop Res, 2004. 22(4): p. 910-7.
35. Pappo, A. S. and U. Dirksen, Rhabdomyosarcoma, Ewing Sarcoma, and Other Round Cell Sarcomas. J Clin Oncol, 2018. 36(2): p. 168-179.
36. Debes, J. D., et al., p300 in prostate cancer proliferation and progression. Cancer Res, 2003. 63(22): p. 7638-40.
37. Liao, Z. W., et al., High expression of p300 is linked to aggressive features and poor prognosis of nasopharyngeal carcinoma. J Transl Med, 2012. 10: p. 110.
38. Li, M., et al., High expression of transcriptional coactivator p300 correlates with aggressive features and poor prognosis of hepatocellular carcinoma. J Transl Med, 2011. 9: p. 5.
39. Jaffe, N., et al., Improved outlook for Ewing's sarcoma with combination chemotherapy (vincristine, actinomycin D and cyclophosphamide) and radiation therapy. Cancer, 1976. 38(5): p. 1925-30.
40. Granowetter, L., et al., Dose-intensified compared with standard chemotherapy for nonmetastatic Ewing sarcoma family of tumors: a Children's Oncology Group Study. J Clin Oncol, 2009. 27(15): p. 2536-41.
41. Hustu, H. O., D. Pinkel, and C. B. Pratt, Treatment of clinically localized Ewing's sarcoma with radiotherapy and combination chemotherapy. Cancer, 1972. 30(6): p. 1522-7.
42. Ladenstein, R., et al., Primary disseminated multifocal Ewing sarcoma: results of the Euro-EWING 99 trial. J Clin Oncol, 2010. 28(20): p. 3284-91.
43. Wagner, M. J., et al., Vincristine, Ifosfamide, and Doxorubicin for Initial Treatment of Ewing Sarcoma in Adults. Oncologist, 2017. 22(10): p. 1271-1277.
44. Mascarenhas, L., et al., Pilot Study of Adding Vincristine, Topotecan, and Cyclophosphamide to Interval-Compressed Chemotherapy in Newly Diagnosed Patients With Localized Ewing Sarcoma: A Report From the Children's Oncology Group. Pediatr Blood Cancer, 2016. 63(3): p. 493-8.
45. Raciborska, A., et al., Vincristine, irinotecan, and temozolomide in patients with relapsed and refractory Ewing sarcoma. Pediatr Blood Cancer, 2013. 60(10): p. 1621-5.
46. Attia, S., et al., A phase II trial of regorafenib in patients with advanced Ewing sarcoma and related tumors of soft tissue and bone: SARC024 trial results. Cancer Med, 2023. 12(2): p. 1532-1539.
47. Celik, H., et al., Clofarabine inhibits Ewing sarcoma growth through a novel molecular mechanism involving direct binding to CD99. Oncogene, 2018. 37(16): p. 2181-2196.
48. Grohar, P. J., et al., Ecteinascidin 743 interferes with the activity of EWS-FLI1 in Ewing sarcoma cells. Neoplasia, 2011. 13(2): p. 145-53.
49. Meyers, P. A., et al., Open-Label, Multicenter, Phase I/I1, First-in-Human Trial of TK216: A First-Generation EWS::FLI1 Fusion Protein Antagonist in Ewing Sarcoma. J Clin Oncol, 2024: p. JC02400020.
50. Grohar, P., et al., SARC037: Results of phase I study of trabectedin given as a 1-hour (h) infusion in combination with low dose irinotecan in relapsed/refractory Ewing sarcoma (ES). 2023, American Society of Clinical Oncology.
51. Ansari, M. S. Z., et al., Pharmacological targeting of CBP/p300 drives a redox/autophagy axis leading to senescence-induced growth arrest in non-small cell lung cancer cells. Cancer Gene Ther, 2023. 30(1): p. 124-136.
52. Waddell, A., et al., Pharmacological Inhibition of CBP/p300 Blocks Estrogen Receptor Alpha (ERalpha) Function through Suppressing Enhancer H3K27 Acetylation in Luminal Breast Cancer. Cancers (Basel), 2021. 13(11).
53. Wang, R., et al., Targeting Lineage-specific MITF Pathway in Human Melanoma Cell Lines by A-485, the Selective Small-molecule Inhibitor of p300/CBP. Mol Cancer Ther, 2018. 17(12): p. 2543-2550.
54. Prieur, A., et al., p53 and p16(INK4A) independent induction of senescence by chromatin-dependent alteration of S-phase progression. Nat Commun, 2011. 2: p. 473.
55. Sen, P., et al., Histone Acetyltransferase p300 Induces De Novo Super-Enhancers to Drive Cellular Senescence. Mol Cell, 2019. 73(4): p. 684-698 e8.
56. Cho, J., et al., Ewing sarcoma gene Ews regulates hematopoietic stem cell senescence.

Blood, 2011. 117(4): p. 1156-66.
57. Li, H., et al., Ewing sarcoma gene EWS is essential for meiosis and B lymphocyte development. J Clin Invest, 2007. 117(5): p. 1314-23.
58. Matsunobu, T., et al., The possible role of EWS-Fli1 in evasion of senescence in Ewing family tumors. Cancer Res, 2006. 66(2): p. 803-11.
59. Ban, J., et al., EWS-FLI1 suppresses NOTCH-activated p53 in Ewing's sarcoma. Cancer Res, 2008. 68(17): p. 7100-9.
60. Hu, H. M., et al., EWS/FLI1 suppresses retinoblastoma protein function and senescence in Ewing's sarcoma cells. J Orthop Res, 2008. 26(6): p. 886-93.
61. Deneen, B. and C. T. Denny, Loss of p16 pathways stabilizes EWS/FLI1 expression and complements EWS/FLI1 mediated transformation. Oncogene, 2001. 20(46): p. 6731-41.
62. Freund, A., et al., Lamin B1 loss is a senescence-associated biomarker. Mol Biol Cell, 2012. 23(11): p. 2066-75.
63. Gil, J. and G. Peters, Regulation of the INK4b-ARF-INK4a tumour suppressor locus: all for one or one for all. Nat Rev Mol Cell Biol, 2006. 7(9): p. 667-77.
64. Shimi, T., et al., The role of nuclear lamin B1 in cell proliferation and senescence. Genes Dev, 2011. 25(24): p. 2579-93.
65. Childs, B. G., et al., Senescence and apoptosis: dueling or complementary cell fates? EMBO Rep, 2014. 15(11): p. 1139-53.
66. Antonescu, C. R., et al., Sarcomas With CIC-rearrangements Are a Distinct Pathologic Entity With Aggressive Outcome: A Clinicopathologic and Molecular Study of 115 Cases. Am J Surg Pathol, 2017. 41(7): p. 941-949.
67. Zhou, X. R., et al., P300/CBP inhibition sensitizes mantle cell lymphoma to PI3Kdelta inhibitor idelalisib. Acta Pharmacol Sin, 2022. 43(2): p. 457-469.
68. Yang, Y., et al., Discovery of Highly Potent, Selective, and Orally Efficacious p300/CBP Histone Acetyltransferases Inhibitors. J Med Chem, 2020. 63(3): p. 1337-1360.
69. Morrison-Smith, C. D., et al., Combined Targeting of the BRD4-NUT-p300 Axis in NUT Midline Carcinoma by Dual Selective Bromodomain Inhibitor, NE02734. Mol Cancer Ther, 2020. 19(7): p. 1406-1414.
70. Welti, J., et al., Targeting the p300/CBP Axis in Lethal Prostate Cancer. Cancer Discov, 2021. 11(5): p. 1118-1137.
71. Friedman, J., T. Hastie, and R. Tibshirani, Regularization Paths for Generalized Linear Models via Coordinate Desc ent. Journal of statistical software. 33(1): p. 1-22.
72. Marsh G P, et al., Development of p300-targeting degraders with enhanced selectivity and onset of degradation. RSC Med Chem. 2025; 16(5):2049-60.

Example 3. The following can illustrate representative pharmaceutical dosage forms, containing a p300/CBP inhibitor or a pharmaceutically acceptable salt thereof and/or a senotherapeutic (e.g., a senolytic or senomorphic) or a pharmaceutically acceptable salt thereof for therapeutic or prophylactic use in humans. The tablets can optionally comprise an enteric coating.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| Compound X= (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | |
| (pH adjustment to 7.0-7.5) | q.s. |
| Water for injection | q.s. ad 1 mL |
| (v) Injection 2 (10 mg/mL) | mg/mL |
| Compound X= (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | |
| (pH adjustment to 7.0-7.5) | q.s. |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The United States Publication US 2022/0288032 is hereby incorporated by reference in its entirety. The invention has been described with reference to various specific and preferred embodiments and techniques. However, it should be understood that many variations and modifications may be made while remaining within the spirit and scope of the invention

1. A method for treating cancer in an animal in need thereof, comprising administering (e.g., co-administering) to the animal a p300/CBP (p300 and/or CBP) modulator or a pharmaceutically acceptable salt thereof and a senotherapeutic (e.g., a senolytic or senomorphic) or a pharmaceutically acceptable salt thereof.

2. A pharmaceutical composition or pharmaceutical combination, comprising a p300/CBP (p300 and/or CBP) modulator or a pharmaceutically acceptable salt thereof and a senotherapeutic (e.g., a senolytic or senomorphic) or a pharmaceutically acceptable salt thereof.

3. The method of claim 1, wherein the p300/CBP (p300 and/or CBP) modulator is a p300/CBP (p300 and/or CBP) inhibitor.

4. The method of claim 1, wherein the p300/CBP (p300 and/or CBP) modulator is a p300/CBP (p300 and/or CBP) degrader.

5. The method of claim 1, wherein the p300/CBP (p300 and/or CBP) modulator is a p300/CBP (p300 and/or CBP) histone acetyltransferase (HAT) inhibitor or bromodomain inhibitor.

6. The method of claim 1, wherein the p300/CBP (p300 and/or CBP) modulator or a pharmaceutically acceptable salt thereof is B029-2, XP-524, A-485, CCS1477, NE02734, B026, FT-7051 or a compound (e.g., p300/CBP inhibitor) or a pharmaceutically acceptable salt thereof as described in WO 2016/044770.

7. The method of claim 1, wherein the p300/CBP (p300 and/or CBP) modulator (e.g., inhibitor) is a compound of formula (I) (iP300w): 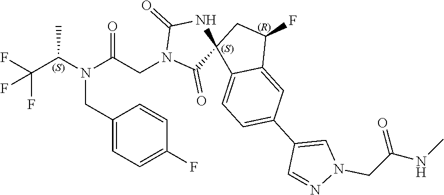
or a pharmaceutically acceptable salt thereof to the animal.

8. The method of claim 1, wherein the p300/CBP (p300 and/or CBP) modulator or a pharmaceutically acceptable salt thereof is a p300/CBP (p300 and/or CBP) degrader.

9. The method of claim 1, wherein the p300/CBP (p300 and/or CBP) modulator or a pharmaceutically acceptable salt thereof is a PROTAC.

10. The method of claim 9, wherein the PROTAC is BT-02C, dCBP-1, or JQAD1.

11. The method of claim 1, wherein the senotherapeutic or a pharmaceutically acceptable salt thereof is Dasatinib, Fisetin, Navitoclax, or Quercetin.

12. The method of claim 1, wherein the senotherapeutic is a tyrosine kinase inhibitor (TKI) or a pharmaceutically acceptable salt thereof, selected from the group consisting of Dasatinib, Imatinib, Nilotinib, Bosutinib, Ponatinib, or Asciminib, Sunitinib, Sorafenib, Axitinib, Lenvatinib, Gefitinib, Erlotinib, Vandetanib, Cabozantinib, Pazopanib, Crizotinib, Ceritinib, Brigatinib, Larotrectinib, Entrectinib, Regorafenib, Osimertinib, Alectinib, Trametinib, Neratinib, and Tucatinib.

13. The method of claim 1, wherein the senotherapeutic is a PI3K (phosphoinositide 3-kinase) inhibitor or a pharmaceutically acceptable salt thereof, selected from the group consisting of Idelalisib, Alpelisib, Copanlisib, Duvelisib, Buparlisib, Pictilisib, Apitolisib, Dactolisib, Taselisib, Omipalisib, Voxtalisib.

14. The method of claim 1, wherein the cancer is a cancer driven by an oncogene that is dependent on p300/CBP (p300 and/or CBP) activity (e.g., the method to reduce the proliferation of oncogene-driven cancer cells).

15. The method of claim 14, wherein the oncogene is EWSR1::FLI1, EWSR1::ERG, AML::ETO, ATXN1::DUX4, CIC::DUX4, CIC::FOXO4, CIC::NUTM1, CIC::LEUTX, MYB::NFIB, RUNX1::ETO, PAX3::FOXO1, PAX3::FOXO4, PAX3::INO80D, PAX3::AFX, PAX3::NCOA1, PAX3::NCOA2, BCOR::CCNB3, BCOR::MAML3, BCOR::ITD, PAX7::FOXO1, ASPSCR1::TFE3, EWSR1::CREB1, EWSR1::ATF1, PAX3::MAML3, MECT1::MAML2, BRD4::NUTM1, BRD3::NUTM1, NSD3::NUTM1, YWHAE::NUTM2, EWSR1::WT1, EP300::BCOR, FUS::ERG, FUS::CREB3L1, FUS::CREB3L2a, EWSR1::CREB3L1, EWSR1::CREB3L2, FUS::CREB3L2, FUS::DDIT3a, NAB2::STAT6, VGLL2::CITED2, SS18::SSX1, SS18::SSX2, SS18::SSX4, SS18L1::SSX1, BCOR::MAML3, NUTM2A::CIC, YWHAE::NUTM2B, EWSR1::SP3, PAX8::PPARG, RUNX1::ET, TMPRSS2::ERG, TMPRSS2::ETV1, or TMPRSS2::ETV4.

16. The method of claim 1, wherein the cancer is a pediatric sarcoma, CIC-rearranged sarcoma, Ewing sarcoma, or CIC-DUX4 sarcoma.

17. A pharmaceutical composition or pharmaceutical combination comprising a p300/CBP (p300 and/or CBP) modulator or a pharmaceutically acceptable salt thereof and a senotherapeutic (e.g., a senolytic or senomorphic) or a pharmaceutically acceptable salt thereof for medical treatment.

18. The use of a pharmaceutical composition or pharmaceutical combination comprising a p300/CBP (p300 and/or CBP) modulator or a pharmaceutically acceptable salt thereof and a senotherapeutic (e.g., a senolytic or senomorphic) or a pharmaceutically acceptable salt thereof for the treatment of cancer.

19. (canceled)
